SEQUENCE LISTING (102 sequences)

SEQ ID NO: 1 - DNA, 257 bp, Homo sapiens
gtgagtctatgggacccttgatgttttctttccccttcttttctatggttaagttcatgtcataggaaggggagaagtaacagggtatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 2 - protein, 146 aa, Homo sapiens
VHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 3 - DNA, 19 bp, Artificial
  Synthetic guide strand-shRNA miR#1
ctcctcaggagtcagatgc

SEQ ID NO: 4 - DNA, 19 bp, Artificial
  Synthetic guide strand-shRNA miR#1mod
tcaggagtcaggtgcgcgc

SEQ ID NO: 5 - DNA, 20 bp, Artificial
  Synthetic guide strand-shRNA miR#2
agacttctcctcaggagtca

SEQ ID NO: 6 - DNA, 21 bp, Artificial
  Synthetic guide strand-shRNA miR#3
tcagtgtggcaaaggtgccct

SEQ ID NO: 7 - DNA, 21 bp, Artificial
  Synthetic guide strand-shRNA miR#3mod
tgtggcaaaggtgccctgcgc

SEQ ID NO: 8 - DNA, 21 bp, Artificial
  Synthetic guide strand-shRNA miR#4
ataacagcatcaggagtggac

SEQ ID NO: 9 - DNA, 21 bp, Artificial
  Synthetic guide strand-shRNA miR#4mod
cagcatcaggagtggacgcgc

SEQ ID NO: 10 - DNA, 21 bp, Artificial
  Synthetic guide strand-shRNA miR#5
ttcatccacgttcaccttgcc

SEQ ID NO: 11 - DNA, 21 bp, Artificial
  Synthetic guide strand-shRNA miR#5mod
tccacgttcaccttgccgcgc

SEQ ID NO: 12 - DNA, 21 bp, Artificial
  Synthetic guide strand-shRNA miR#6
caaagaacctctgggtccaag

SEQ ID NO: 13 - DNA, 21 bp, Artificial
  Synthetic guide strand-shRNA miR#6mod
gaacctctgggtccaaggcgc

SEQ ID NO: 14 - DNA, 21 bp, Artificial
  Synthetic guide strand-shRNA miR#7
ctttcttgccatgagccttca

SEQ ID NO: 15 - DNA, 21 bp, Artificial
  Synthetic guide strand-shRNA miR#7mod
cttgccatgagccttcagcgc

SEQ ID NO: 16 - DNA, 20 bp, Artificial
  Synthetic guide strand-shRNA miR#8
tgaagttctcaggatccacg

SEQ ID NO: 17 - DNA, 21 bp, Artificial
  Synthetic guide strand-shRNA miR#9
ttctttgccaaagtgatgggc

SEQ ID NO: 18 - DNA, 21 bp, Artificial
  Synthetic guide strand-shRNA miR#10
aaaggcaccgagcactttctt

SEQ ID NO: 19 - DNA, 19 bp, Artificial
  Synthetic guide strand-shRNA miR#11
ccagggcctcaccaccaac

SEQ ID NO: 20 - DNA, 19 bp, Artificial
  Synthetic guide strand-shRNA miR#12
ctccacaggagtcagatgc

SEQ ID NO: 21 - DNA, 19 bp, Artificial
  Synthetic guide strand-shRNA miR#12mod
acaggagtcaggtgcgcgc

SEQ ID NO: 22 - DNA, 20 bp, Artificial
  Synthetic guide strand-shRNA miR#13
agacttctccacaggagtca

SEQ ID NO: 23 - DNA, 21 bp, Homo sapiens
aaggtgaaggctcatggcaag

SEQ ID NO: 24 - DNA, 21 bp, Homo sapiens
aagaaagtgctcggtgccttt

SEQ ID NO: 25 - DNA, 15 bp, Artificial
  Synthetic loop
ctccatgtggtagag

SEQ ID NO: 26 - DNA, 53 bp, Artificial
  Synthetic shRNA miR#1
gcatctgactcctgaggagctccatgtggtagagctcctcaggagtcagatgc

SEQ ID NO: 27 - DNA, 53 bp, Artificial
  Synthetic shRNA miR#1mod
gcgcgcacctgactcctgactccatgtggtagagtcaggagtcaggtgcgcgc

SEQ ID NO: 28 - DNA, 55 bp, Artificial
  Synthetic shRNA miR#2
tgactcctgaggagaagtctctccatgtggtagagagacttctcctcaggagtca

SEQ ID NO: 29 - DNA, 57 bp, Artificial
  Synthetic shRNA miR#3
agggcacctttgccacactgactccatgtggtagagtcagtgtggcaaaggtgccct

SEQ ID NO: 30 - DNA, 57 bp, Artificial
  Synthetic shRNA miR#3mod
gcgcagggcacctttgccacactccatgtggtagagtgtggcaaaggtgccctgcgc

SEQ ID NO: 31 - DNA, 57 bp, Artificial
  Synthetic shRNA miR#4
gtccactcctgatgctgttatctccatgtggtagagataacagcatcaggagtggac

SEQ ID NO: 32 - DNA, 57 bp, Artificial
  Synthetic shRNA miR#4mod
gcgcgtccactcctgatgctgctccatgtggtagagcagcatcaggagtggacgcgc

SEQ ID NO: 33 - DNA, 57 bp, Artificial
  Synthetic shRNA miR#5
ggcaaggtgaacgtggatgaactccatgtggtagagttcatccacgttcaccttgcc

SEQ ID NO: 34 - DNA, 57 bp, Artificial
  Synthetic shRNA miR#5mod
gcgcggcaaggtgaacgtggactccatgtggtagagtccacgttcaccttgccgcgc

SEQ ID NO: 35 - DNA, 57 bp, Artificial
  Synthetic shRNA miR#6
cttggacccagaggttctttgctccatgtggtagagcaaagaacctctgggtccaag

SEQ ID NO: 36 - DNA, 57 bp, Artificial
  Synthetic shRNA miR#6mod
gcgccttggacccagaggttcctccatgtggtagaggaacctctgggtccaaggcgc

SEQ ID NO: 37 - DNA, 57 bp, Artificial
  Synthetic shRNA miR#7
tgaaggctcatggcaagaaagctccatgtggtagagctttcttgccatgagccttca

SEQ ID NO: 38 - DNA, 57 bp, Artificial
  Synthetic shRNA miR#7mod
gcgctgaaggctcatggcaagctccatgtggtagagcttgccatgagccttcagcgc

SEQ ID NO: 39 - DNA, 57 bp, Artificial
  Synthetic shRNA miR#8
acgtggatcctgagaacttcactccatgtggtagagtgaagttctcaggatccacgt

SEQ ID NO: 40 - DNA, 57 bp, Artificial
  Synthetic shRNA miR#9
gcccatcactttggcaaagaactccatgtggtagagttctttgccaaagtgatgggc

SEQ ID NO: 41 - DNA, 57 bp, Artificial
  Synthetic shRNA miR#10
aagaaagtgctcggtgcctttctccatgtggtagagaaaggcaccgagcactttctt

SEQ ID NO: 42 - DNA, 53 bp, Artificial
  Synthetic shRNA miR#11
gttggtggtgaggccctggctccatgtggtagagccagggcctcaccaccaac

SEQ ID NO: 43 - DNA, 53 bp, Artificial
  Synthetic shRNA miR#12
gcatctgactcctgtggagctccatgtggtagagctccacaggagtcagatgc

SEQ ID NO: 44 - DNA, 53 bp, Artificial
  Synthetic shRNA miR#12mod
gcgcgcacctgactcctgtctccatgtggtagagacaggagtcaggtgcgcgc

SEQ ID NO: 45 - DNA, 55 bp, Artificial
  Synthetic shRNA miR#13
tgactcctgtggagaagtctctccatgtggtagagagacttctccacaggagtca

SEQ ID NO: 46 - DNA, 100 bp, Artificial
  Synthetic miR#1
cctggcctcctgcagtgccacgctgcatctgactcctgaggagctccatgtggtagagctcctcaggagtcagatgcagtgcggcacatgcttaccagct

SEQ ID NO: 47 - DNA, 100 bp, Artificial
  Synthetic miR#1mod
cctggcctcctgcagtgccacgctgcgcgcacctgactcctgactccatgtggtagagtcaggagtcaggtgcgcgcagtgcggcacatgcttaccagct

SEQ ID NO: 48 - DNA, 102 bp, Artificial
  Synthetic miR#2
cctggcctcctgcagtgccacgcttgactcctgaggagaagtctctccatgtggtagagagacttctcctcaggagtcaagtgcggcacatgcttaccagct

SEQ ID NO: 49 - DNA, 104 bp, Artificial
  Synthetic miR#3
cctggcctcctgcagtgccacgctagggcacctttgccacactgactccatgtggtagagtcagtgtggcaaaggtgccctagtgcggcacatgcttaccagct

SEQ ID NO: 50 - DNA, 104 bp, Artificial
  Synthetic miR#3mod
cctggcctcctgcagtgccacgctgcgcagggcacctttgccacactccatgtggtagagtgtggcaaaggtgccctgcgcagtgcggcacatgcttaccagct

SEQ ID NO: 51 - DNA, 104 bp, Artificial
  Synthetic miR#4
cctggcctcctgcagtgccacgctgtccactcctgatgctgttatctccatgtggtagagataacagcatcaggagtggacagtgcggcacatgcttaccagct

SEQ ID NO: 52 - DNA, 104 bp, Artificial
  Synthetic miR#4mod
cctggcctcctgcagtgccacgctgcgcgtccactcctgatgctgctccatgtggtagagcagcatcaggagtggacgcgcagtgcggcacatgcttaccagct

SEQ ID NO: 53 - DNA, 104 bp, Artificial
  Synthetic miR#5
cctggcctcctgcagtgccacgctggcaaggtgaacgtggatgaactccatgtggtagagttcatccacgttcaccttgccagtgcggcacatgcttaccagct

SEQ ID NO: 54 - DNA, 104 bp, Artificial
  Synthetic miR#5mod
cctggcctcctgcagtgccacgctgcgcggcaaggtgaacgtggactccatgtggtagagtccacgttcaccttgccgcgcagtgcggcacatgcttaccagct

SEQ ID NO: 55 - DNA, 104 bp, Artificial
  Synthetic miR#6
cctggcctcctgcagtgccacgctcttggacccagaggttctttgctccatgtggtagagcaaagaacctctgggtccaagagtgcggcacatgcttaccagct

SEQ ID NO: 56 - DNA, 104 bp, Artificial
  Synthetic miR#6mod
cctggcctcctgcagtgccacgctgcgccttggacccagaggttcctccatgtggtagaggaacctctgggtccaaggcgcagtgcggcacatgcttaccagct

SEQ ID NO: 57 - DNA, 104 bp, Artificial
  Synthetic miR#7
cctggcctcctgcagtgccacgcttgaaggctcatggcaagaaagctccatgtggtagagctttcttgccatgagccttcaagtgcggcacatgcttaccagct

SEQ ID NO: 58 - DNA, 104 bp, Artificial
  Synthetic miR#7mod
cctggcctcctgcagtgccacgctgcgctgaaggctcatggcaagctccatgtggtagagcttgccatgagccttcagcgcagtgcggcacatgcttaccagct

SEQ ID NO: 59 - DNA, 104 bp, Artificial
  Synthetic miR#8
cctggcctcctgcagtgccacgctacgtggatcctgagaacttcactccatgtggtagagtgaagttctcaggatccacgtagtgcggcacatgcttaccagct

SEQ ID NO: 60 - DNA, 104 bp, Artificial
  Synthetic miR#9
cctggcctcctgcagtgccacgctgcccatcactttggcaaagaactccatgtggtagagttctttgccaaagtgatgggcagtgcggcacatgcttaccagct

SEQ ID NO: 61 - DNA, 104 bp, Artificial
  Synthetic miR#10
cctggcctcctgcagtgccacgctaagaaagtgctcggtgcctttctccatgtggtagagaaaggcaccgagcactttcttagtgcggcacatgcttaccagct

SEQ ID NO: 62 - DNA, 100 bp, Artificial
  Synthetic miR#11
cctggcctcctgcagtgccacgctgttggtggtgaggccctggctccatgtggtagagccagggcctcaccaccaacagtgcggcacatgcttaccagct

SEQ ID NO: 63 - DNA, 100 bp, Artificial
  Synthetic miR#12
cctggcctcctgcagtgccacgctgcatctgactcctgtggagctccatgtggtagagctccacaggagtcagatgcagtgcggcacatgcttaccagct

SEQ ID NO: 64 - DNA, 100 bp, Artificial
  Synthetic miR#12mod
cctggcctcctgcagtgccacgctgcgcgcacctgactcctgtctccatgtggtagagacaggagtcaggtgcgcgcagtgcggcacatgcttaccagct

SEQ ID NO: 65 - DNA, 102 bp, Artificial
  Synthetic miR#13
cctggcctcctgcagtgccacgcttgactcctgtggagaagtctctccatgtggtagagagacttctccacaggagtcaagtgcggcacatgcttaccagct

SEQ ID NO: 66 - DNA, 357 bp, Artificial
  Synthetic bAS3-int2/miR#1
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcatctgactcctgaggagctccatgtggtagagctcctcaggagtcagatgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 67 - DNA, 357 bp, Artificial
  Synthetic bAS3-int2/miR#1mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcgcacctgactcctgactccatgtggtagagtcaggagtcaggtgcgcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 68 - DNA, 359 bp, Artificial
  Synthetic bAS3-int2/miR#2
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgcttgactcctgaggagaagtctctccatgtggtagagagacttctcctcaggagtcaagtgcggcacatgcttaccagcttccagctaccattctgcttttattttatggttgggataaggctggattattctgagtccaagctaggcccttttgctaatcatgttcatacctcttatcttcctcccacag

SEQ ID NO: 69 - DNA, 361 bp, Artificial
  Synthetic bAS3-int2/miR#3
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctagggcacctttgccacactgactccatgtggtagagtcagtgtggcaaaggtgccctagtgcggcacatgcttaccagcttccagctaccattctgcttttattttatggttgggataaggctggattattctgagtccaagctaggcccttttgctaatcatgttcatacctcttatcttcctcccacag

SEQ ID NO: 70 - DNA, 361 bp, Artificial
  Synthetic bAS3-int2/miR#3mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcagggcacctttgccacactccatgtggtagagtgtggcaaaggtgccctgcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 71 - DNA, 361 bp, Artificial
  Synthetic bAS3-int2/miR#4
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgtccactcctgatgctgttatctccatgtggtagagataacagcatcaggagtggacagtgcggcacatgcttaccagcttccagctaccattctgcttttattttatggttgggataaggctggattattctgagtccaagctaggcccttttgctaatcatgttcatacctcttatcttcctcccacag

SEQ ID NO: 72 - DNA, 361 bp, Artificial
  Synthetic bAS3-int2/miR#4mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcgtccactcctgatgctgctccatgtggtagagcagcatcaggagtggacgcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 73 - DNA, 361 bp, Artificial
  Synthetic bAS3-int2/miR#5
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctggcaaggtgaacgtggatgaactccatgtggtagagttcatccacgttcaccttgccagtgcggcacatgcttaccagcttccagctaccattctgcttttattttatggttgggataaggctggattattctgagtccaagctaggcccttttgctaatcatgttcatacctcttatcttcctcccacag

SEQ ID NO: 74 - DNA, 361 bp, Artificial
  Synthetic bAS3-int2/miR#5mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcggcaaggtgaacgtggactccatgtggtagagtccacgttcaccttgccgcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 75 - DNA, 361 bp, Artificial
  Synthetic bAS3-int2/miR#6
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctcttggacccagaggttctttgctccatgtggtagagcaaagaacctctgggtccaagagtgcggcacatgcttaccagcttccagctaccattctgcttttattttatggttgggataaggctggattattctgagtccaagctaggcccttttgctaatcatgttcatacctcttatcttcctcccacag

SEQ ID NO: 76 - DNA, 361 bp, Artificial
  Synthetic bAS3-int2/miR#6mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgccttggacccagaggttcctccatgtggtagaggaacctctgggtccaaggcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 77 - DNA, 361 bp, Artificial
  Synthetic bAS3-int2/miR#7
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgcttgaaggctcatggcaagaaagctccatgtggtagagctttcttgccatgagccttcaagtgcggcacatgcttaccagcttccagctaccattctgcttttattttatggttgggataaggctggattattctgagtccaagctaggcccttttgctaatcatgttcatacctcttatcttcctcccacag

SEQ ID NO: 78 - DNA, 361 bp, Artificial
  Synthetic bAS3-int2/miR#7mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgctgaaggctcatggcaagctccatgtggtagagcttgccatgagccttcagcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 79 - DNA, 361 bp, Artificial
  Synthetic bAS3-int2/miR#8
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctacgtggatcctgagaacttcactccatgtggtagagtgaagttctcaggatccacgtagtgcggcacatgcttaccagcttccagctaccattctgcttttattttatggttgggataaggctggattattctgagtccaagctaggcccttttgctaatcatgttcatacctcttatcttcctcccacag

SEQ ID NO: 80 - DNA, 361 bp, Artificial
  Synthetic bAS3-int2/miR#9
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcccatcactttggcaaagaactccatgtggtagagttctttgccaaagtgatgggcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 81 - DNA, 361 bp, Artificial
  Synthetic bAS3-int2/miR#10
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctaagaaagtgctcggtgcctttctccatgtggtagagaaaggcaccgagcactttcttagtgcggcacatgcttaccagcttccagctaccattctgcttttattttatggttgggataaggctggattattctgagtccaagctaggcccttttgctaatcatgttcatacctcttatcttcctcccacag

SEQ ID NO: 82 - DNA, 357 bp, Artificial
  Synthetic bAS3-int2/miR#11
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgttggtggtgaggccctggctccatgtggtagagccagggcctcaccaccaacagtgcggcacatgcttaccagcttccagctaccattctgcttttattttatggttgggataaggctggattattctgagtccaagctaggcccttttgctaatcatgttcatacctcttatcttcctcccacag

SEQ ID NO: 83 - DNA, 357 bp, Artificial
  Synthetic bAS3-int2/miR#12
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcatctgactcctgtggagctccatgtggtagagctccacaggagtcagatgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 84 - DNA, 357 bp, Artificial
  Synthetic bAS3-int2/miR#12mod
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgcgcacctgactcctgtctccatgtggtagagacaggagtcaggtgcgcgcagtgcggcacatgcttaccagcttccagctaccattctgcttttattttatggttgggataaggctggattattctgagtccaagctaggcccttttgctaatcatgttcatacctcttatcttcctcccacag

SEQ ID NO: 85 - DNA, 359 bp, Artificial
  Synthetic bAS3-int2/miR#13
gtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgcttgactcctgtggagaagtctctccatgtggtagagagacttctccacaggagtcaagtgcggcacatgcttaccagcttccagctaccattctgcttttattttatggttgggataaggctggattattctgagtccaagctaggcccttttgctaatcatgttcatacctcttatcttcctcccacag

SEQ ID NO: 86 - DNA, 2051 bp, Artificial
  Synthetic modified  AS3 sequence (5′-3′) + ( AS3-int2/miR#7mod)
acatttgcttctgacacaactgtgttcactagcaacctcaaacagacaccatggtgcacctgactcctgaggagaagtctgccgttactgccctgtgggacaaggtgaacgtggatgccgttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaagtcaaagcccacggtaaaaaggtgctcggtgcctttagtgatggcctggctcacctggacaacctcaagggcacctttgcccagctgagtgagctgcactgtgacaagctgcacgtggatcctgagaacttcagggtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctgcgctgaaggctcatggcaagctccatgtggtagagcttgccatgagccttcagcgcagtgcggcacatgcttaccagcttccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatggtacgtaccgataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatccttt

SEQ ID NO: 87 - DNA, 2051 bp, Artificial
  Synthetic modified  AS3 sequence (5′-3′) + ( AS3-int2/miR#10)
acatttgcttctgacacaactgtgttcactagcaacctcaaacagacaccatggtgcacctgactcctgaggagaagtctgccgttactgccctgtgggacaaggtgaacgtggatgccgttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaaaaaggttctgggcgctttcagtgatggcctggctcacctggacaacctcaagggcacctttgcccagctgagtgagctgcactgtgacaagctgcacgtggatcctgagaacttcagggtgagtctatgggacccttgatgttttctttccccttcttttctatggttaagttcatgtcataggaaggggagaagtaacagggtatttctgcatataaattgtaactgatgtaagaggtttcatattgctaatagcagctacaacctggcctcctgcagtgccacgctaagaaagtgctcggtgcctttctccatgtggtagagaaaggcaccgagcactttcttagtgcggcacatgcttaccagcttccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatggtacgtaccgataaaattttgaattttgtaatttgtttttgtaattctttagtttgtatgtctgttgctattatgtctactattctttcccctgcactgtaccccccaatccccccttttcttttaaaagttaaccgataccgtcgagatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatccttt

SEQ ID NO: 88 - DNA, 26 bp, Artificial
  Synthetic probe
cctgtcatgcccacacaaatctctcc

SEQ ID NO: 89 - DNA, 22 bp, Artificial
  Synthetic primer
gctgtcatctcttgtgggctgt

SEQ ID NO: 90 - DNA, 21 bp, Artificial
  Synthetic primer
actcatgggagctgctggttc

SEQ ID NO: 91 - DNA, 19 bp, Artificial
  Synthetic probe
cgcacggcaagaggcgagg

SEQ ID NO: 92 - DNA, 20 bp, Artificial
  Synthetic primer
tcccccgcttaatactgacg

SEQ ID NO: 93 - DNA, 20 bp, Artificial
  Synthetic primer
caggactcggcttgctgaag

SEQ ID NO: 94 - DNA, 17 bp, Artificial
  Synthetic primer
agggcacctttgcccag

SEQ ID NO: 95 - DNA, 22 bp, Artificial
  Synthetic primer
gccaccactttctgataggcag

SEQ ID NO: 96 - DNA, 18 bp, Artificial
  Synthetic primer
aagggcacctttgccaca

SEQ ID NO: 97 - DNA, 22 bp, Artificial
  Synthetic primer
gccaccactttctgataggcag

SEQ ID NO: 98 - DNA, 21 bp, Artificial
  Synthetic primer
cggtcaacttcaagctcctaa

SEQ ID NO: 99 - DNA, 20 bp, Artificial
  Synthetic primer
acagaagccaggaacttgtc

SEQ ID NO: 100 - DNA, 4 bp, Artificial
  Synthetic modified cap
gcgc

SEQ ID NO: 101 - RNA, 100 nt, Artificial
  Synthetic amiR of the present invention (Figure 1B)
  misc_feature (25)..(43): n is a, c, g, or u
  misc_feature (59)..(77): n is a, c, g, or u
ccuggccuccugcagugccacgcunnnnnnnnnnnnnnnnnnncuccaugugguagagnnnnnnnnnnnnnnnnnnnagugcggcacaugcuuaccagcu

SEQ ID NO: 102 - DNA, 223 bp, Homo sapiens
  HBB-exon2 (Figure 1D)
cctgaagttctcaggatccacgtgcagcttgtcacagtgcagctcactcagtgtggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagc

(19) United States
(12) Patent Application Publication
(10) Pub. No.: US 2023/0357769 A1
(43) Pub. Date: Nov. 9, 2023

(54) BIFUNCTIONAL LENTIVIRAL VECTORS ALLOWING THE BS-GLOBIN SILENCING AND EXPRESSION OF AN ANTI-SICKLING HBB AND USES THEREOF FOR GENE THERAPY OF SICKLE CELL DISEASE

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); FONDATION IMAGINE, Paris (FR); UNIVERSITÉ D'EVRY VAL D'ESSONNE, Evry (FR); UNIVERSITÉ DE PARIS, Paris (FR)

(72) Inventors: Annarita MICCIO, Paris (FR); Fulvio MAVILIO, Modena (IT); Mario AMENDOLA, Evry (FR); Marina CAVAZZANA, Paris (FR); Megane BRUSSON, Paris (FR)

(21) Appl. No.: 18/246,270

(22) PCT Filed: Sep. 21, 2021
(86) PCT No.: PCT/EP2021/075887

§ 371 Date: Mar. 22, 2023

(30) Foreign Application Priority Data
Sep. 22, 2020 (EP) 20306075.1

(51) Int. Cl. C12N 15/113; C12N 15/86; A61K 35/28; A61P 7/06

(52) U.S. Cl. CPC C12N 15/113; C12N 15/86; A61K 35/28; A61P 7/06; C12N 2310/141; C12N 2310/531; C12N 2740/15043

(57) ABSTRACT

Gene therapy of SCO is based on the transplantation of genetically modified HSCs. Several LV approaches based on gene addition consist in transducing patient HSCs with a lentiviral vector expressing an anti-sickling β-like globin chain such as use of β^{AS3} HBB anti-sickling variants. Here, the inventors have improved the design of the LV-AS3 vector to treat SCO patients. These LVs allow the simultaneous expression of the potent anti-sickling β^{AS3}-globin and an artificial miR (amiR) silencing the β^{S}-globin. The reduction of β^{S}-globin levels will increase the incorporation of β^{AS3}-globin in Hb tetramers, which should increase the proportion of corrected RBCs in SCO patients. The inventors selected the best-performing miRs, and modified the therapeutic β^{AS3}-globin transgene by inserting silent mutations to avoid the recognition by the amiR and the silencing of the transgene.

### FIELD OF THE INVENTION

The present invention is in the field of medicine, in particular haematology and gene therapy.

### BACKGROUND OF THE INVENTION

Sickle cell disease (SCD) is an autosomal-recessive genetic disorder that affects 312,000 newborns worldwide every year. It is caused by a point mutation at codon 6 of the HBB globin gene (GAG→GTG), which leads at the protein level to the substitution of a hydrophilic glutamic acid residue by a hydrophobic valine residue (Glu→Val) resulting in the production of the sickle β globin (β^{S}). Together with the two a chains, the two β^{S} chains form an abnormal adult Hb: the sickle Hb (HbS, α₂β^{S}^{₂}). Under hypoxic conditions, the valine interacts with a hydrophobic pocket on the surface of a second HbS tetramer and induces the polymerization of the HbS tetramers. HbS polymerization leads to the formation of sickle shaped RBCs, which are less deformable, more fragile and associated with a dramatically decreased lifespan in blood circulation (approximately 20-40 days) (Franco et al., 2006). Importantly, sickle RBCs tend to obstruct small vessels. These abnormal sickle shaped RBCs are responsible for chronic hemolytic anemia, painful vaso-occlusive crises and progressive organ failure leading to a high mortality rate in these patients (Sundd et al., 2018). For example, the spleen of patients with SCD is prematurely damaged by vaso-occlusions. The spleen then no longer completely fulfills its role in the control of certain bacterial infections thus increasing the risk of serious infections.

The clinical severity of SCD is alleviated by the co-inheritance of genetic mutations causing a sustained fetal γ-globin chain production at adult age, a condition termed hereditary persistence of fetal Hb (HPFH). Indeed SCD-HPFH patients with HbF levels of >20% have less severe disease phenotype and improved survival (Powars et al., 1984). The γ-globin exerts an anti-sickling effect in SCD by displacing the β^{S}-globin from the HbS to form HbF (α₂γ₂) and a hybrid tetramer (α₂γβ^{S}) that cannot polymerize with others HbS molecules (Akinsheye et al., 2011).

The therapeutic care of SCD is largely based on the prevention of complications (hydroxyurea, prevention of infections) blood transfusion in case of acute anemia, RBC exchange (removal of abnormal RBCs and transfusion of healthy RBCs) and symptomatic treatments (painkillers). However, long-term treatments are very expensive and remains associated with a poor quality of life for patients. The hydroxyurea pharmacological treatment is used to increase HbF levels in patient' cells but is not effective in all patients and often cannot reach a sufficient HbF expression to completely rescue the phenotype. The only curative and definitive treatment currently available is allogenic HSC transplantation but its implementation is limited by the lack of compatible HLA (Human leukocyte antigen) donors and the associated immunological risks (i.e., graft versus host reaction).

Gene therapy of SCD is based on the transplantation of genetically modified HSCs and has several advantages over allogenic HSC transplantation. Indeed, there is no need to find a suitable donor and therefore it avoids the risk of toxicity of graft versus host disease Moreover, for the same reasons it avoids the need for immunosuppressive treatments. Many strategies have been developed to modify patients HSCs in order to rescue the SCD phenotype. These strategies are based on the use of lentiviral vectors (LV) and genome editing technologies such as the clustered regularly interspaced short palindromic repeat (CRISPR)-associated nuclease Cas9 system (CRISPR/Cas9). LVs are largely used in gene therapy as they allow stable integration of the gene of interest even in non-diving cells (such as HSCs) with a very low oncogenic risk thanks to its safe integration profile. In addition, clinical studies based on LV approaches can be readily implemented, whereas genome editing strategies still require extensive validation to be translated into the clinical realm.

Several LV approaches based on gene addition consist in transducing patient HSCs with a lentiviral vector expressing an anti-sickling β-like globin chain (the natural anti-sickling fetal γ-globin gene or β^{A-T87Q} and β^{AS3} HBB anti-sickling variants). The LVs express the transgene under the control of the β-globin promoter and critical elements of the LCR (Locus control region) HS (DNase-hypersensitive sites) enhancers, which are regulatory elements in the HBB locus that boost the expression of the β-globin and β-like globin genes (Cavazzana et al., 2017). These LVs allows the expression of the transgene restricted to the erythroid lineage. In the phase ½ HGB-205 study conducted in France, 3 SCD patients who had recurrent vaso-occlusive events were treated with the BB305 vector expressing the β^{A-T87Q} variant that impairs the formation of lateral contacts necessary for HbS polymerization. BM HSCs of the 3 patients were harvested twice and then they underwent myeloablation by IV infusion of busulfan prior to the infusion of genetically modified HSCs. The first SCD patient (patient 1204 [β^{S}/β^{S}]) was successfully treated. Fifteen months after HSCs transplantation, the vector copy number (VCN) in vivo was around 2 and the proportion of the HbAT87Q was about 50% of the total hemoglobins (Ribeil et al., 2017). The two other SCD patients (patient 1207 [β^{S}/β^{S}] and patient 1208 [β^{S}/β⁰]) received a smaller dose of cells than patient 1204 and the VCN in the pre-transplant product was also lower. The level of transduction in the peripheral blood was stable but lower than that observed for patient 1204. In consequence, only patient 1208 became symptoms free due to moderate HbAT87Q levels associated with good levels of the endogenous HbF, while no clinical benefit was observed for patient 1207. In the phase 1/2 HGB-206 trial, they used the same vector as in the HGB-205 trial and similar results were obtained with no benefits in patients who received a low dose of BM cells with a low VCN (<1) but encouraging results in patients who received higher dose of BM cells with higher VCN (>1) (Cavazzana et al., 2017; Magrin et al., 2019). Therefore, the LV-mediated expression of a therapeutic β-globin is promising as it was able to correct the phenotype of some SCD patients, but LVs still need some improvements to rescue the phenotype of patients that receive a lower dose of HSCs with a low VCN.

LVs approaches have also been used to treat β-thalassemia which is an another β-hemoglobinopathy characterized by a reduced or absent β-globin expression. The most severe form of β-thalassemias require regular blood transfusion in order to increase HbA levels. In an Italian clinical trial, the GLOBE vector has been tested in 4 pediatric β-thalassemic patients who were transfusion-dependent. GLOBE vector expresses the wild type human β-globin under the control of the β globin promoter and the HS2 HS3 elements from the LCR. Twelve months after transplantation, the mean VCN in erythroid precursors was 0.58 (range 0.1-1.97); three of the 4 patients became transfusion-independent and the transfusion requirement was reduced by 20% in the fourth patient (Marktel et al., 2019). As GLOBE LV can transduce a good proportion of HSCs and drive high levels of HBB expression it has been adapted by our group to treat SCD by replacing the wild type β-globin transgene with the β^{AS3} HBB anti-sickling variant, which contains mutations leading to 3 the G16D, E22A and T87Q amino acids substitutions. A22 and Q87 impair, respectively, the axial and lateral contacts necessary for the formation of HbS polymers, and D16 increases the affinity to α-globin chains. In pre-clinical studies, the LV-AS3 vector showed a high transduction efficiency in patient' HSCs resulting in a strong expression of the β^{AS3}-globin in RBCs cultured in vitro and reduction of 34% of the sickling in vitro assay at a VCN of 1.7 (Poletti et al., 2018; Weber et al., 2018). However, the sickle RBC phenotype was only partially corrected, indicating that classical gene replacement strategy is hampered by the high expression level of the competing endogenous β^{S}-globin. These data suggest that amelioration of the LV-AS3 vector design is required to improve its therapeutic efficacy.

Another LV-based gene therapy aims to increase the levels of endogenous HbF by reversing the fetal-to-adult Hb switch. To this aim, Brendel et al. developed the BCH_BB-LCRshRNA(miR) vector that expresses a microRNA-adapted shRNA (shRNAmiR) targeting BCL11A which is an endogenous transcriptional repressor of the HBG genes (Brendel et al., 2016; Sankaran et al., 2008). The shRNAmiR is under the control of the β-globin promoter and the HS2 and HS3 elements of the LCR. Therefore, the expression of the shRNAmiR is restricted to the erythroid lineage thus avoiding the known negative impact of BCL11A knockdown in others hematopoietic lineages (Luc et al., 2016; Yu et al., 2012). In preclinical studies, this vector showed good results in terms of HbF induction and reversion of the SCD phenotype (Brendel et al., 2016). This strategy of reversing the fetal-to-adult hemoglobin switch by interfering with the transcriptional repressor BCL11A is currently tested in clinical trial and showed encouraging results. Indeed, 7-15 months after HSC transplantation, two SCD subjects showed around 70% of HbF⁺ cells and a HbF/(HbF+HbS) ratio of 24 to 32% (Esrick et al., 2019).

Therefore, additional improvements in LV design are required to obtain a robust therapy of SCD.

### SUMMARY OF THE INVENTION

The present invention is defined by the claims. In particular, the present invention relates to bifunctional lentiviral vectors allowing the β^{S}-globin silencing and expression of an anti-sickling HBB and uses thereof for gene therapy of sickle cell disease.

### DETAILED DESCRIPTION OF THE INVENTION

Strategies based on gene addition of therapeutic β-like globin using a LV showed good results in SCD patients who received a drug product with a VCN>1 and when the VCN in vivo was around 2. Unfortunately obtaining a VCN>1 is not always achievable even with optimized protocols and poses some concerns on the low but still possible genotoxicity due to the integration of a high number of vector copies in hematopoietic cells. In addition, Weber et al showed that despite a high transduction efficiency obtained with the LV-AS3 vector, the SCD RBC phenotype derived is only partially corrected in in vitro sickling assay. This is probably due to HbS levels that remain high in these cells. Therefore, gene addition strategies using a LV still require improvements to fully correct the SCD phenotype and to show a benefit in patients for which a VCN>1 cannot be obtained.

Here, the inventors have improved the design of the LV-AS3 vector to treat SCD patients. In particular, they developed new LVs combining two strategies: gene addition and silencing. These LVs allow the simultaneous expression of the potent anti-sickling β^{AS3}-globin and an artificial miR (amiR) silencing the β^{S}-globin. The reduction of β^{S}-globin levels will increase the incorporation of β^{AS3}-globin in Hb tetramers, which should increase the proportion of corrected RBCs in SCD patients. The inventors opted for a miR-based strategy instead of other RNA interference system such as short hairpin RNAs (shRNAs) to reduce the potential toxicity. shRNAs mimic the structure of the pre-miR and can be functional in human cells but they might interfere with the miR processing machinery within the cell due to their high expression levels. miR-based gene therapies are considered safer as miRs are naturally present in human cells. The inventors selected the best-performing miRs, and modified the therapeutic β^{AS3}-globin transgene by inserting silent mutations to avoid the recognition by the amiR and the silencing of the transgene.

The first object of the present invention relates to a nucleic acid molecule having the sequence as set forth in SEQ ID NO:1 wherein a sequence encoding for an artificial microRNA (amiR) suitable for reducing the expression of the β^{S}-globin is inserted between the nucleotide at position 85 and the nucleotide at position 86 in SEQ ID NO:1 and/or ii) between the nucleotide at position 146 and the nucleotide at position 147 in SEQ ID NO:1.

>bAS3 intron 2 sequence (5′-3′)
SEQ ID NO: 1
gtgagtctatgggacccttgatgttttctttccccttcttttctatggt
taagttcatgtcataggaaggggagaagtaacagggtatttctgcatat
aaattgtaactgatgtaagaggtttcatattgctaatagcagctacaat
ccagctaccattctgcttttattttatggttgggataaggctggattat
tctgagtccaagctaggcccttttgctaatcatgttcatacctcttatc
ttcctcccacag

As used herein, the term “β-globin” or “HBB” has its general meaning in the art and refers to a globin protein, which along with alpha globin (HBA), makes up the most common form of haemoglobin (Hb) in adult humans. Normal adult human Hb is a heterotetramer consisting of two alpha chains and two beta chains. HBB is encoded by the HBB gene on human chromosome 11. It is 146 amino acids long and has a molecular weight of 15,867 Da. An exemplary human amino acid sequence is represented by SEQ ID NO:2.

>sp|P68871|HBB_HUMAN Hemoglobin subunit beta OS =
Homo sapiens OX = 9606 GN = HBB PE = 1 SV = 2
SEQ ID NO: 2
VHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHV
DPENFRLLGNVLVCVLAHHFGKEFTPPVQAAYOKVVAGVANALAHKYH

As used herein, the term “β^{S}-globin” has its general meaning in the art and refers to the sickled β-globin that is caused by a point mutation at codon 6 of the HBB globin gene (GAG→GTG), which leads at the protein level to the substitution of a hydrophilic glutamic acid residue at position 6 by a hydrophobic valine residue (Glu→Val).

As used herein, the term “microRNA”, “miRNA” or “miR” has its general meaning in the art and refers to a small non-coding RNA molecule (containing around 22 nucleotides) found in plants, animals and some viruses, that functions in RNA silencing and post-transcriptional regulation of gene expression. miRNAs resemble the small interfering RNAs (siRNAs) of the RNA interference (RNAi) pathway, except that miRNAs derive from regions of RNA transcripts that fold back on themselves to form short hairpins, whereas siRNAs derive from longer regions of double-stranded RNA. The miRNAs are first transcribed as primary miRNAs (pri-miRNAs) with caps and a poly-A tail. The pri-miRNAs are then processed into precursor miRNAs (pre-miRNAs) by an enzyme called Drosha. The structure of pre-miRNA is a 70 nucleotide-long stem-loop structure. The pre-miRNAs are then exported into the cytoplasm and split into mature miRNAs by an enzyme called Dicer. These mature miRNAs will integrate into the RNA-induced silencing complex (RISC) and activate the RISC. The activated RISC can then allow miRNAs to bind with the targeted mRNA and silence the gene expression.

As used herein, the term “artificial miRNA”, “artificial miR” or “amiR” refers to a shRNA that is embedded into a miRNA backbone that is derived from a naturally-occurring miRNA. More particularly, the amiR of the present invention consists of a shRNA having 5′ and 3′flanking regions with one or more structural features of a corresponding region of a naturally-occurring miRNA. For example, any miRNAs described in miRBase can be used for providing the miRNA backbone. Mechanistically, the artificial miRNA is first cleaved to produce the shRNA and then cleaved again by DICER to produce siRNA. The siRNA is then incorporated into the RISC for target mRNA degradation.

As used herein, the term “short hairpin RNA” or “shRNA” has its general meaning in the art and refers to a unimolecular RNA that is capable of performing RNA interference and that has a passenger strand, a loop, and a guide strand. Typically, the shRNA of the present invention adopts a stem-loop structure. As used herein, a “stem-loop structure” refers to a nucleic acid having a secondary structure that includes a region of nucleotides which are known or predicted to form a double strand or duplex (stem portion or stem region) that is linked on one side by a region of predominantly single-stranded nucleotides (loop portion or terminal loop region). The terms “hairpin” and “fold-back” structures can also be used to refer to stem-loop structures. Such structures are well known in the art and the term is used consistently with its known meaning in the art. As described herein, the stem region is a region formed by a guide strand and a passenger strand. As described herein, the “guide strand” represents the portion that associates with RISC as opposed to the “passenger strand”, which is not associated with RISC. Typically, the passenger and guide strands are thus substantially complementary to each other. The passenger/guide strand can be about 11 to about 29 nucleotides in length, and more preferably 17 to 19 nucleotides in length.

In some embodiments, the miRNA backbone is derived from miR-223.

As used herein, the term “miR-223” has its general meaning in the art and refers to the miR available from the data base http://mirbase.org under the miRBase accession number MI0000300 (hsa-mir-223).

Typically, the structure of the amiR of the present invention is depicted in FIG. 1B and derives from the structure described in (Amendola et al., 2009. Mechanistically, the artificial miRNA is first cleaved to produce the shRNA and then cleaved again by DICER to produce siRNA. The siRNA is then incorporated into the RISC for target mRNA degradation.

In some embodiments, the sequence encoding for the guide strand consists of a nucleic acid sequence selected from SEQ ID NO:3 to SEQ ID NO:22. Preferably, the sequence encoding for the guide strand consists of the nucleic acid sequence of SEQ ID NO:3 (guide strand-shRNA miR #1), SEQ ID NO:11 (guide strand-shRNA miR #5mod), SEQ ID NO:14 (guide strand-shRNA miR #7), SEQ ID NO:15 (guide strand-shRNA miR #7mod),SEQ ID NO:18 (guide strand-shRNA miR #10) or SEQ ID NO:20 (guide strand-shRNA miR #12). More preferably, the sequence encoding for the guide strand consists of a nucleic acid sequence that is complementary to the nucleic acid sequence as set forth in SEQ ID NO:23 or SEQ ID NO:24 that are comprised in the exon 2 of the nucleic acid encoding for β^{S}-globin as depicted in FIG. 1D. Even more preferably, the sequence encoding for the guide strand consists of the nucleic acid sequence of SEQ ID NO:15 (guide strand-shRNA miR #7mod) or SEQ ID NO:18 (guide strand-shRNA miR #10).

>(guide strand-shRNA miR#1)
SEQ ID NO: 3
CTCCTCAGGAGTCAGATGC
>(guide strand-shRNA miR#1mod)
SEQ ID NO: 4
TCAGGAGTCAGGTGCGCGC
>(guide strand-shRNA miR#2)
SEQ ID NO: 5
AGACTTCTCCTCAGGAGTCA
>(guide strand-shRNA miR#3)
SEQ ID NO: 6
TCAGTGTGGCAAAGGTGCCCT
>(guide strand-shRNA miR#3mod)
SEQ ID NO: 7
TGTGGCAAAGGTGCCCTGCGC
>(guide strand-shRNA miR#4)
SEQ ID NO: 8
ATAACAGCATCAGGAGTGGAC
>(guide strand-shRNA miR#4mod)
SEQ ID NO: 9
CAGCATCAGGAGTGGACGCGC
>(guide strand-shRNA miR#5)
SEQ ID NO: 10
TTCATCCACGTTCACCTTGCC
>(guide strand-shRNA miR#5mod)
SEQ ID NO: 11
TCCACGTTCACCTTGCCGCGC
>(guide strand-shRNA miR#6)
SEQ ID NO: 12
CAAAGAACCTCTGGGTCCAAG
>(guide strand-shRNA miR#6mod)
SEQ ID NO: 13
GAACCTCTGGGTCCAAGGCGC
>(guide strand-shRNA miR#7)
SEQ ID NO: 14
CTTTCTTGCCATGAGCCTTCA
>(guide strand-shRNA miR#7mod)
SEQ ID NO: 15
CTTGCCATGAGCCTTCAGCGC
>(guide strand-shRNA miR#8)
SEQ ID NO: 16
TGAAGTTCTCAGGATCCACGT
>(guide strand-shRNA miR#9)
SEQ ID NO: 17
TTCTTTGCCAAAGTGATGGGC
>(guide strand-shRNA miR#10)
SEQ ID NO: 18
AAAGGCACCGAGCACTTTCTT
>(guide strand-shRNA miR#11)
SEQ ID NO: 19
CCAGGGCCTCACCACCAAC
>(guide strand-shRNA miR#12)
SEQ ID NO: 20
CTCCACAGGAGTCAGATGC
>(guide strand-shRNA miR#12mod)
SEQ ID NO: 21
ACAGGAGTCAGGTGCGCGC
>(guide strand-shRNA miR#13)
SEQ ID NO: 22
AGACTTCTCCACAGGAGTCA

As used herein, the term “complementary” as used herein includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%.

>(region miR#7mod targeted region on HBB_exon2_CDS)
SEQ ID NO: 23
aaggtgaaggctcatggcaag
>(region miR#10 targeted region on HBB_exon2_CDS)
SEQ ID NO: 24
Aagaaagtgctcggtgccttt

In some embodiments, the guide strand that is complementary to the target can contain mismatches. In some embodiments, the guide strand and the passenger strand may have at least one base pair mismatch. In some embodiments, the guide strand and the passenger strand have 2 base pair mismatches, 3 base pair mismatches, 4 base pair mismatches, 5 base pair mismatches, 6 base pair mismatches, 7 base pair mismatches, 8 base pair mismatches, 9 base pair mismatches, 10 base pair mismatches, 11 base pair mismatches, 12 base pair mismatches, 13 base pair mismatches, 14 base pair mismatches or 15 base pair mismatches. In some embodiments, the guide strand and passenger strand have mismatches at no more than ten consecutive base pairs. In some embodiments, at least one base pair mismatch is located at an anchor position. In some embodiments, at least one base pair mismatch is located in a center portion of the stem.

As described herein, the terminal loop region comprises at least 4 nucleotides. The sequence of the loop can include nucleotide residues unrelated to the target. In some embodiments, the loop segment is encoded by the sequence as set forth in SEQ ID NO:25.

>(loop segment)
SEQ ID NO: 25
CTCCATGTGGTAGAG

In some embodiments, the sequence encoding for the shRNA of the present invention is selected from SEQ ID NO:26 to SEQ ID NO:45 wherein the loop of the shRNA is framed. More preferably, the sequence encoding for the shRNA of the present invention is SEQ ID NO:24 (shRNA miR #1), SEQ ID NO:32 (shRNA miR #5mod), SEQ ID NO:33 (shRNA miR #7), SEQ ID NO:36 (shRNA miR #7mod), SEQ ID NO:39 (shRNA miR #10) or SEQ ID NO:44 (shRNA miR #12). More preferably, the sequence encoding for the shRNA of the present invention is SEQ ID NO:38 (shRNA miR #7mod) or SEQ ID NO:41 (shRNA miR #10).

>SEQ ID NO: 26 (shRNA miR#1)

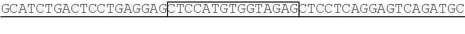

>SEQ ID NO: 27 (shRNA miR#1mod)

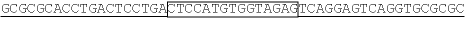

>SEQ ID NO: 28 (shRNA miR#2)

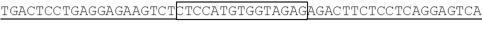

>SEQ ID NO: 29 (shRNA miR#3)

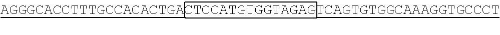

>SEQ ID NO: 30 (shRNA miR#3mod)

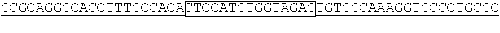

>SEQ ID NO: 31 (shRNA miR#4)

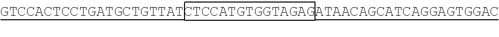

>SEQ ID NO: 32 (shRNA miR#4mod)

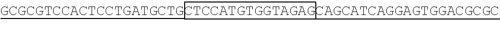

>SEQ ID NO: 33 (shRNA miR#5)

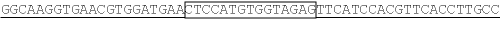

>SEQ ID NO: 34 (shRNA miR#5mod)

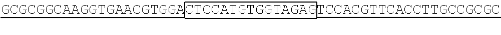

>SEQ ID NO: 35 (shRNA miR#6)

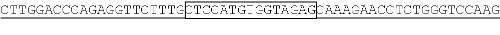

>SEQ ID NO: 36 (shRNA miR#6mod)

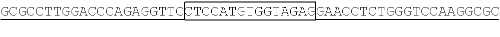

>SEQ ID NO: 37 (shRNA miR#7)

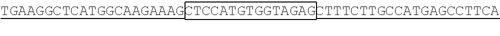

>SEQ ID NO: 38 (shRNA miR#7mod)

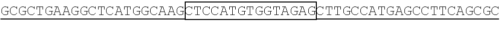

>SEQ ID NO: 39 (shRNA miR#8)

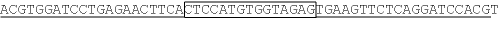

>SEQ ID NO: 40 (shRNA miR#9)

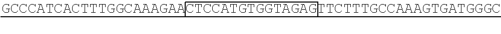

>SEQ ID NO: 41 (shRNA miR#10)

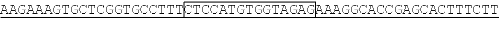

>SEQ ID NO: 42 (shRNA miR#11)

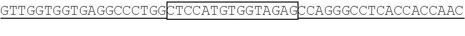

>SEQ ID NO: 43 (shRNA miR#12)

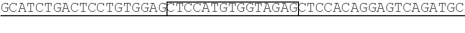

>SEQ ID NO: 44 (shRNA miR#12mod)

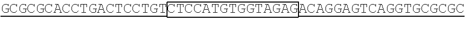

>SEQ ID NO: 45 (shRNA miR#13)

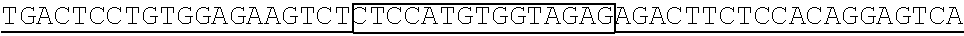

In some embodiments, the sequence encoding for the amiR of the present invention is a sequence selected from SEQ ID NO:46 to SEQ ID NO:65 wherein the sequence of shRNA is underlined and the loop of the amiR is framed. Preferably, the sequence encoding for the amiR of the present invention is SEQ ID NO:56 (miR #1), SEQ ID NO:54 (miR #5mod), SEQ ID NO:57 (miR #7), SEQ ID NO:58 (miR #7mod) SEQ ID NO:61 (miR #10) or SEQ ID NO:63 (miR #12). More preferably, the sequence encoding for the amiR of the present invention is SEQ ID NO:58 (miR #7mod) or SEQ ID NO:61 (miR #10).

>SEQ ID NO: 46 (miR#1)

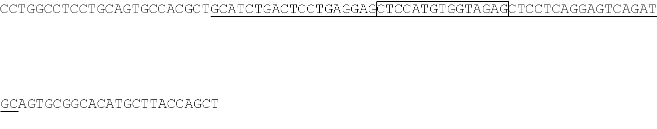

>SEQ ID NO: 47 (miR#1mod)

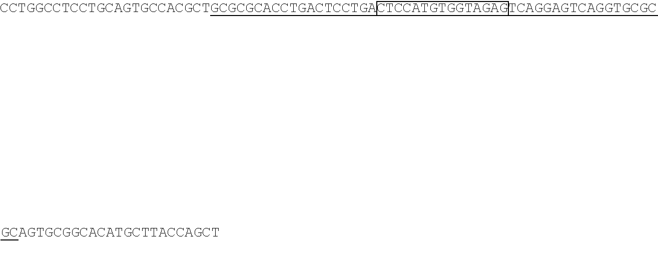

>SEQ ID NO: 48 (miR#2)

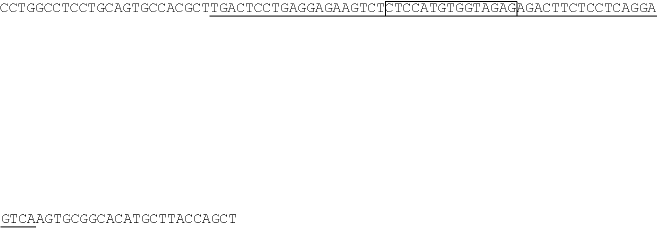

>SEQ ID NO: 49 (miR#3)

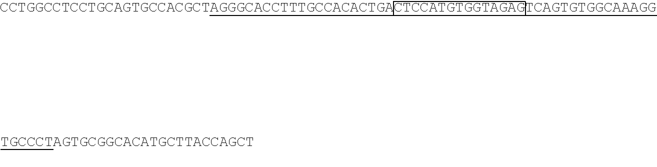

>SEQ ID NO: 50 (miR#3mod)

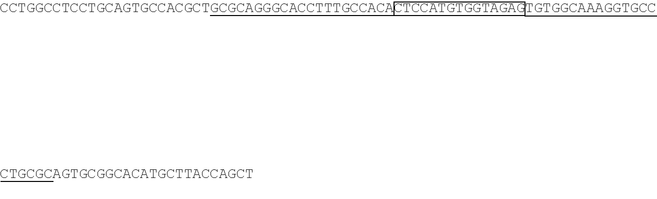

>SEQ ID NO: 51 (miR#4)

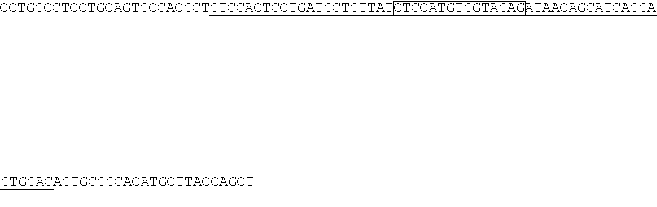

>SEQ ID NO: 52 (miR#4mod)

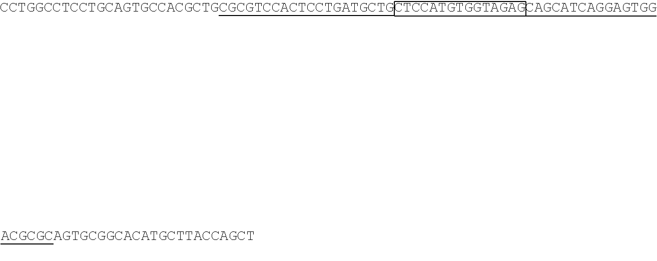

>SEQ ID NO: 53 (miR#5)

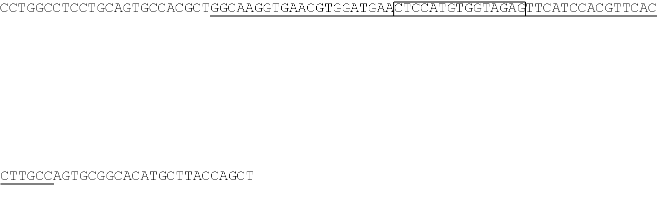

>SEQ ID NO: 54 (miR#5mod)

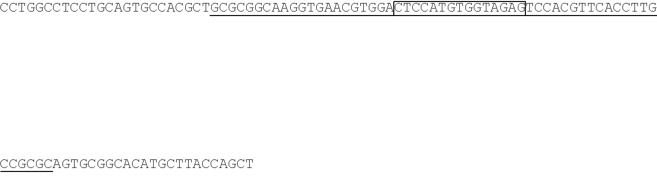

>SEQ ID NO: 55 (miR#6)

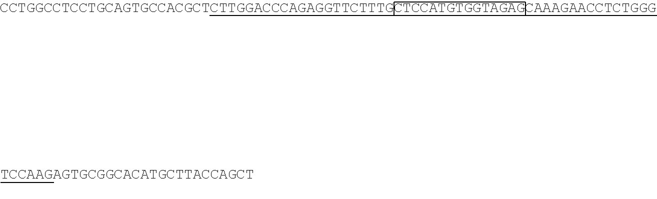

>SEQ ID NO: 56 (miR#6mod)

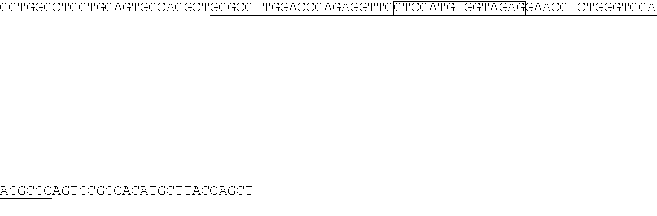

>SEQ ID NO: 57 (miR#7)

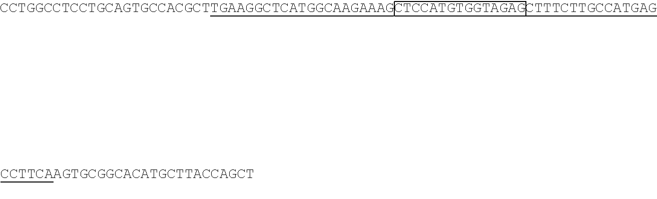

>SEQ ID NO: 58 (miR#7mod)

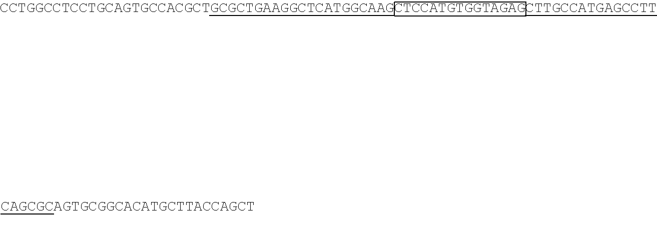

>SEQ ID NO: 59 (miR#8)

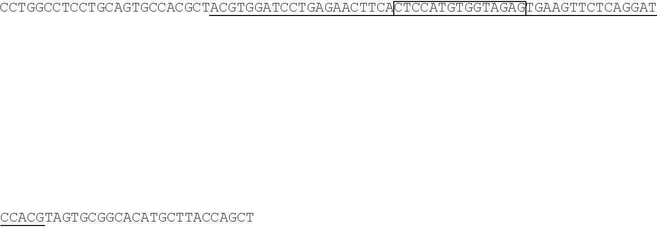

>SEQ ID NO: 60 (miR#9)

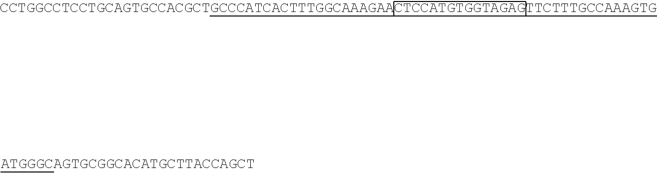

>SEQ ID NO: 61 (miR#10)

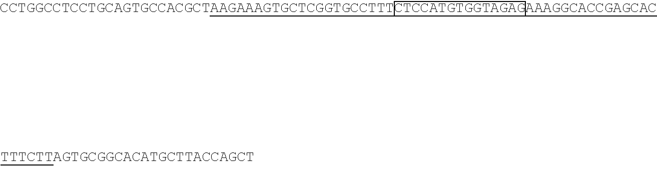

>SEQ ID NO: 62 (miR#11)

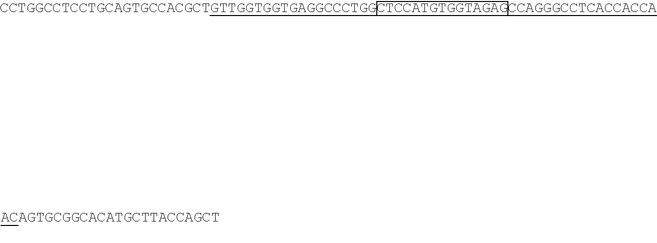

>SEQ ID NO: 63 (miR#12)

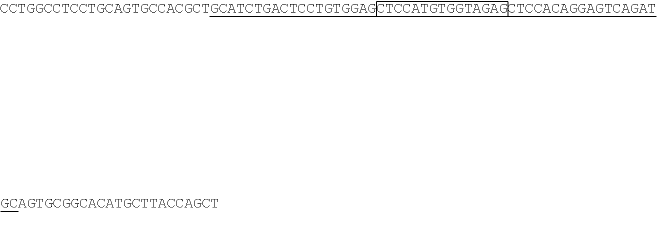

>SEQ ID NO: 64 (miR#12mod)

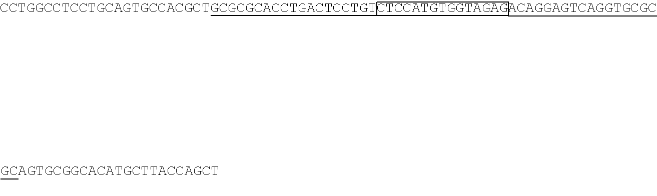

>SEQ ID NO: 65 (miR#13)

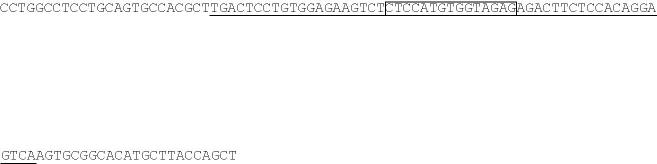

In some embodiments, the nucleic acid molecule of the present invention has a sequence selected from SEQ ID NO:66 to SEQ ID NO:85 wherein the 5′ to 3′ sequence of intron 2 of the βAS3 transgene are in lowercase, the amiR sequence is in uppercase, the sequence of shRNA is underlined and the loop of the amiR is framed. Preferably, the nucleic acid molecule of the present invention has the sequence of SEQ ID NO:66 (βAS3-int2/miR #1), SEQ ID NO:74 (βAS3-int2/miR #5mod), SEQ ID NO:77 (βAS3-int2/miR #7), SEQ ID NO:78 (βAS3-int2/miR #7mod) SEQ ID NO:81 (βAS3-int2/miR #10) or SEQ ID NO:84 (βAS3-int2/miR #12). Preferably, the nucleic acid molecule of the present invention has the sequence of SEQ ID NO:78 (βAS3-int2/miR #7mod) or SEQ ID NO:81 (βAS3-int2/miR #10).

>SEQ ID NO: 66 (βAS3-int2/miR#1)

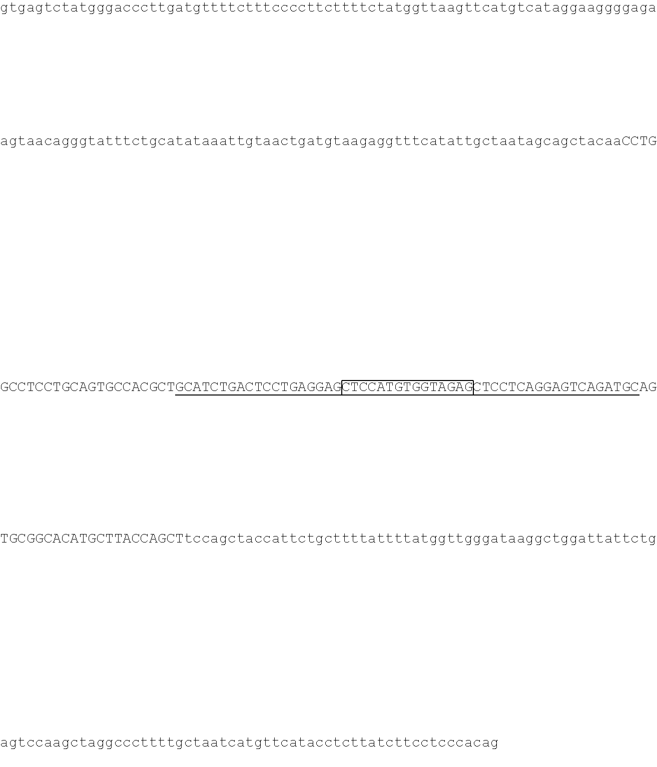

>SEQ ID NO: 67 (βAS3-int2/miR#1mod)

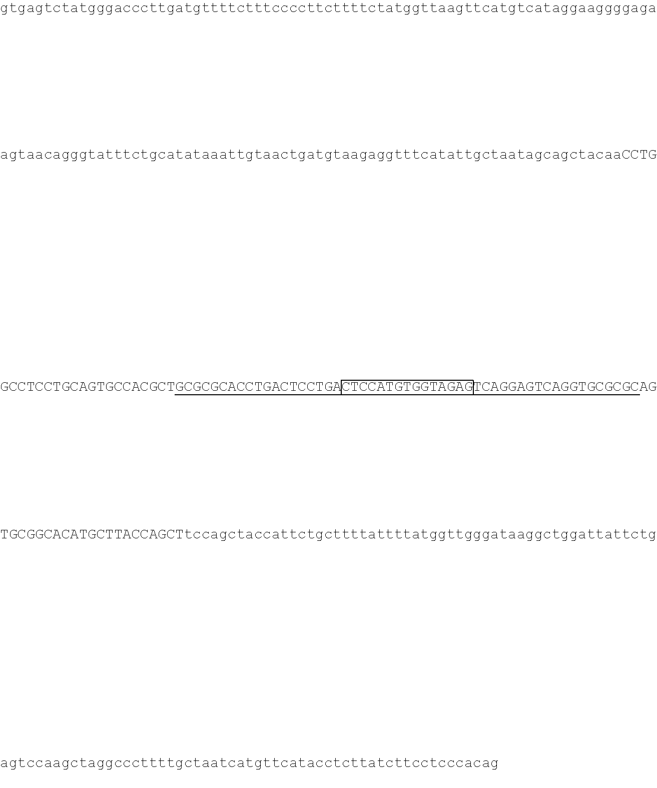

>SEQ ID NO: 68 (βAS3-int2/miR#2)

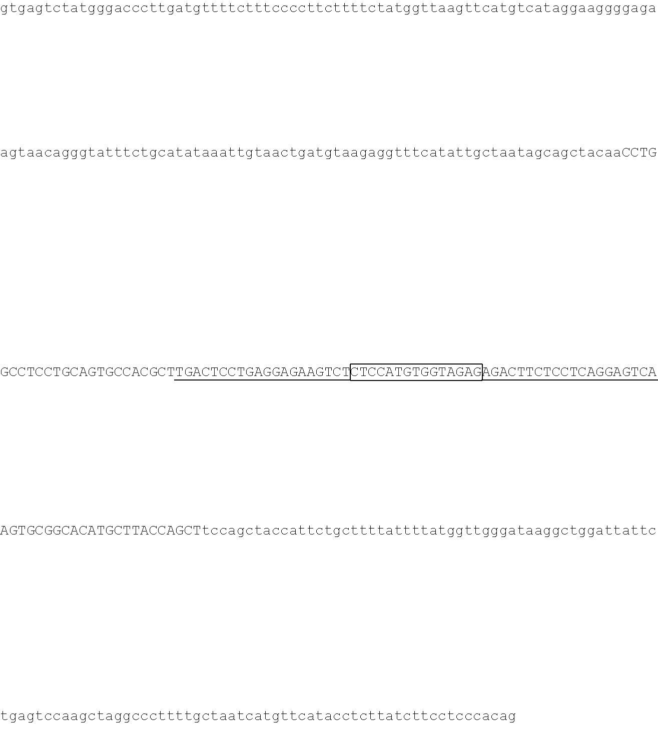

>SEQ ID NO: 69 (βAS3-int2/miR#3)

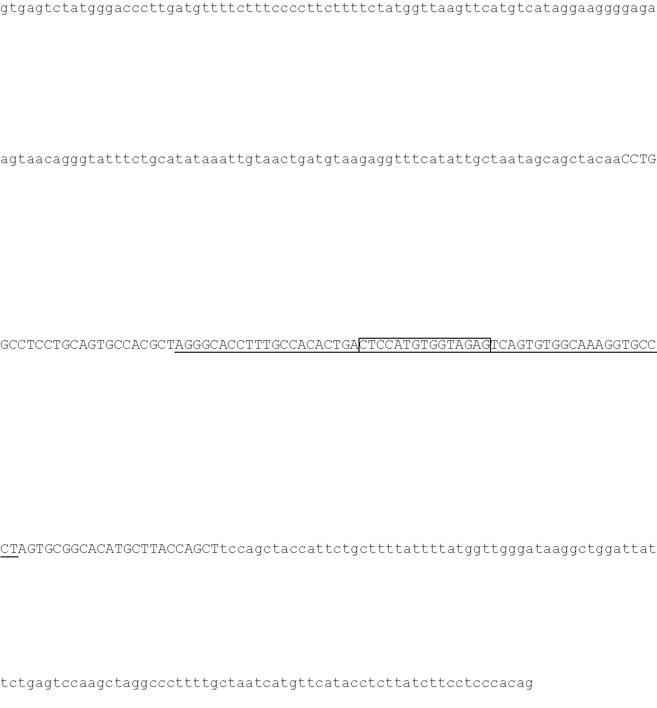

>SEQ ID NO: 70 (βAS3-int2/miR#3mod)

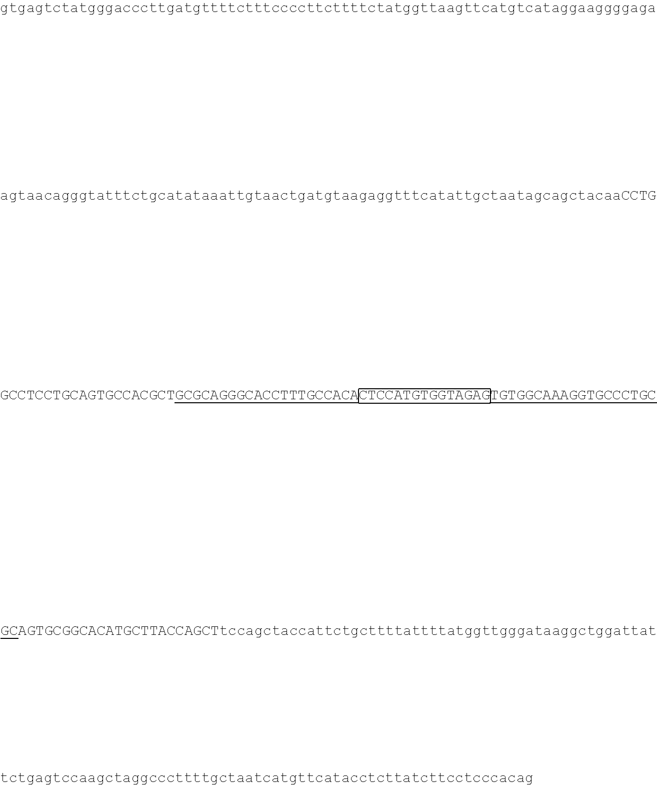

>SEQ ID NO: 71 (βAS3-int2/miR#4)

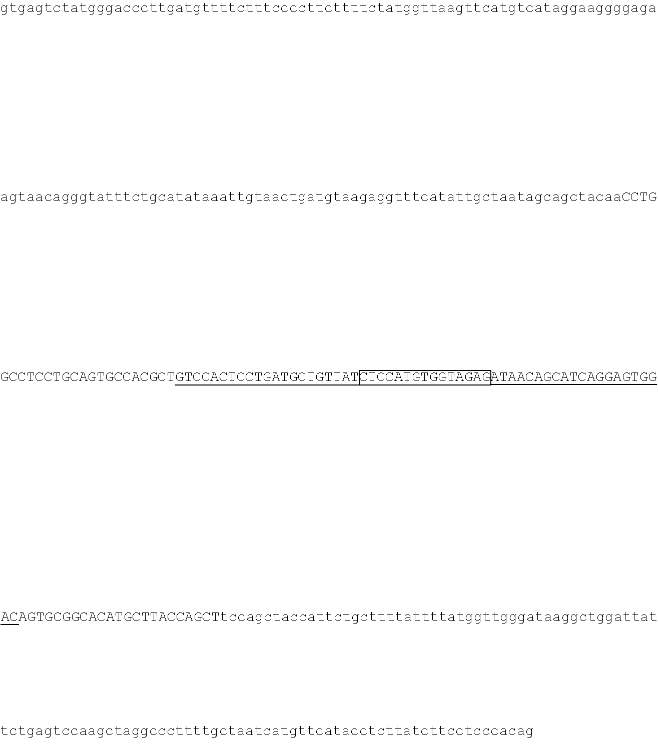

>SEQ ID NO: 72 (βAS3-int2/miR#4mod)

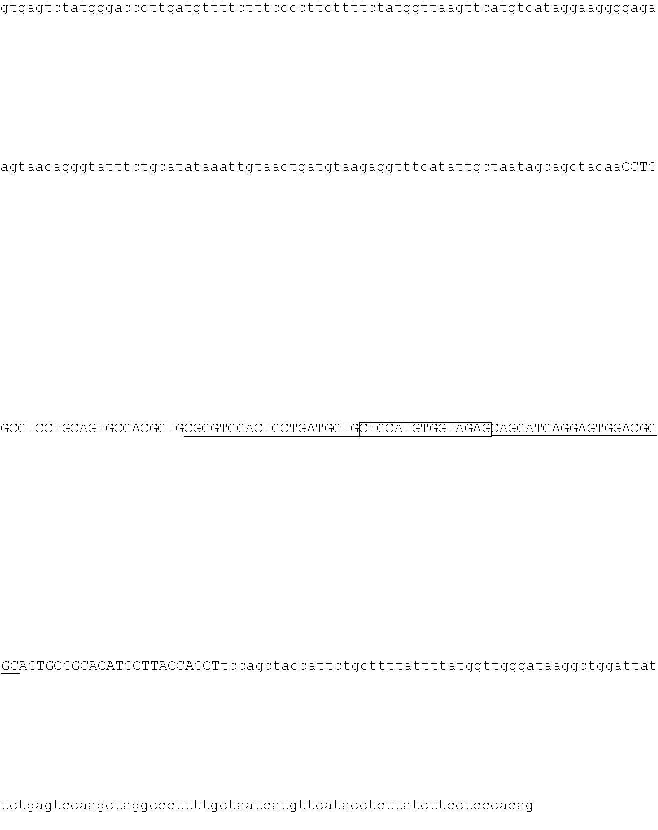

>SEQ ID NO: 73 (βAS3-int2/miR#5)

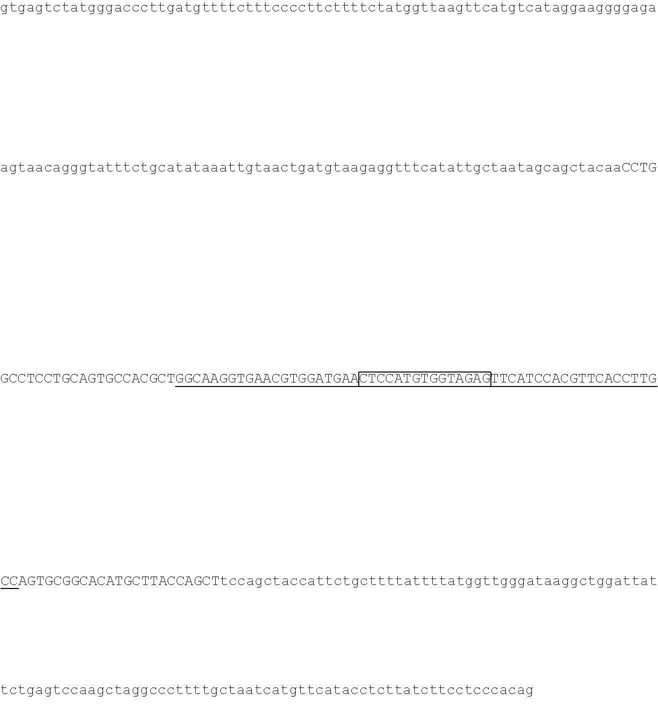

>SEQ ID NO: 74 (βAS3-int2/miR#5mod)

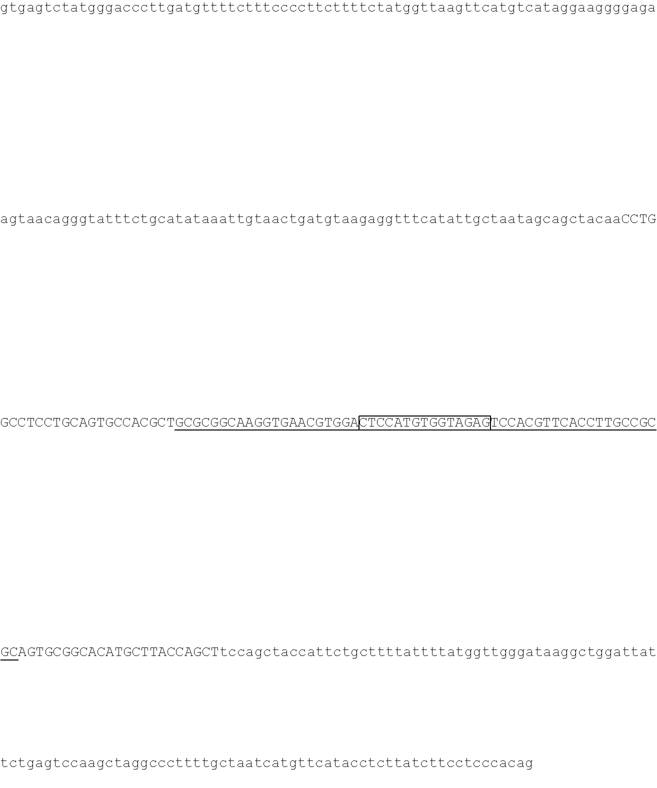

>SEQ ID NO: 75 (βAS3-int2/miR#6)

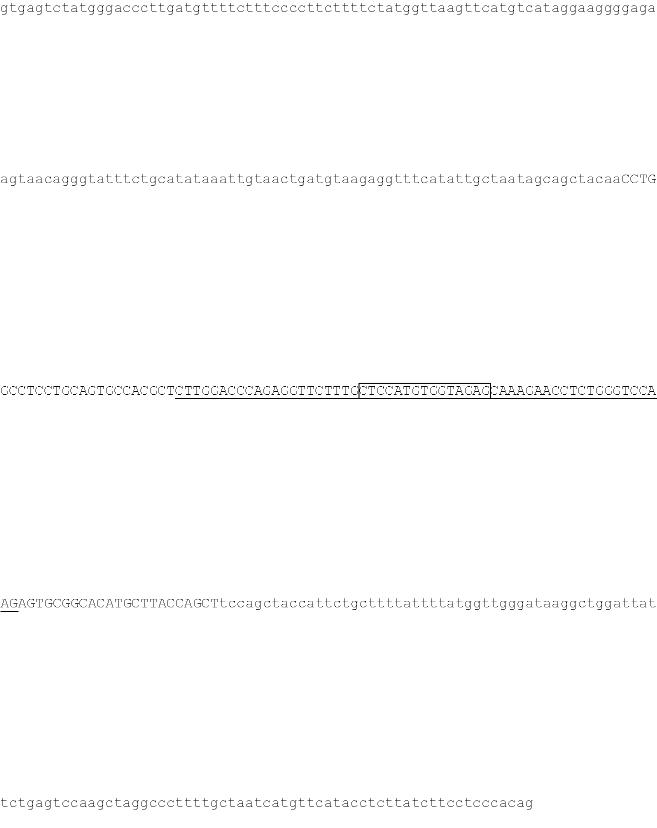

>SEQ ID NO: 76 (βAS3-int2/miR#6mod)

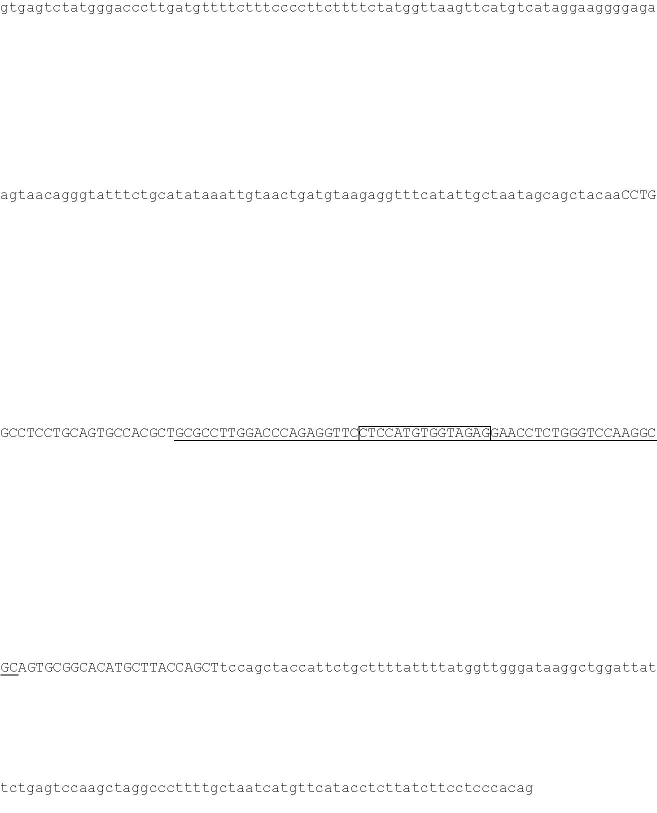

>SEQ ID NO: 77 (βAS3-int2/miR#7)

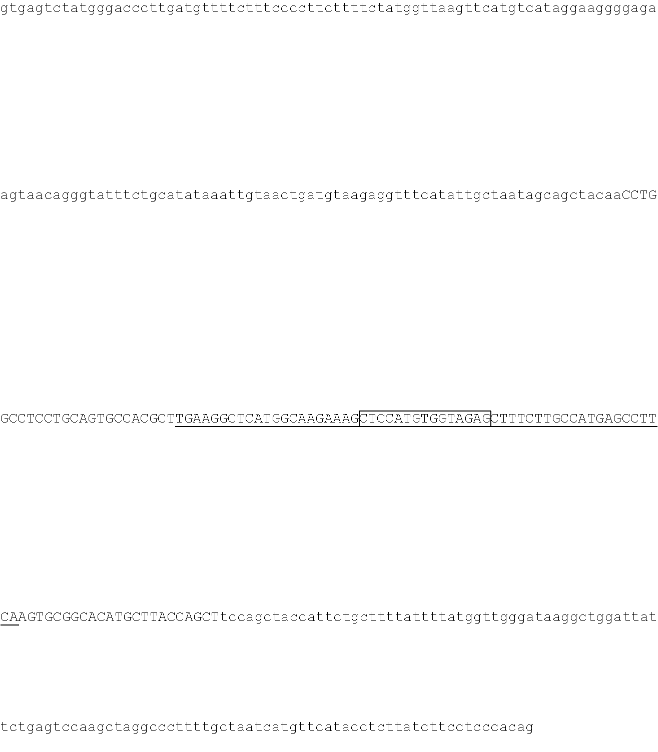

>SEQ ID NO: 78 (βAS3-int2/miR#7mod)

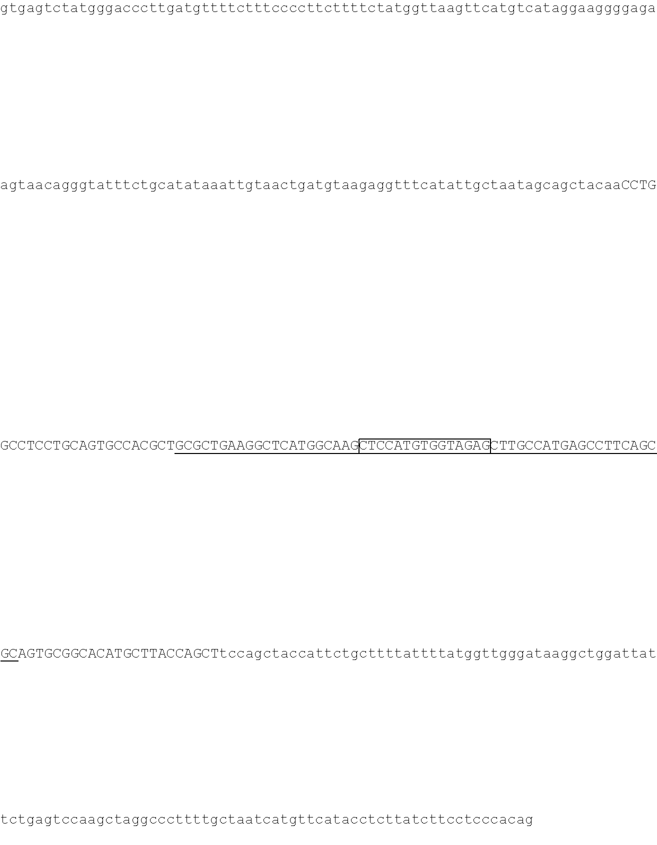

>SEQ ID NO: 79 (βAS3-int2/miR#8)

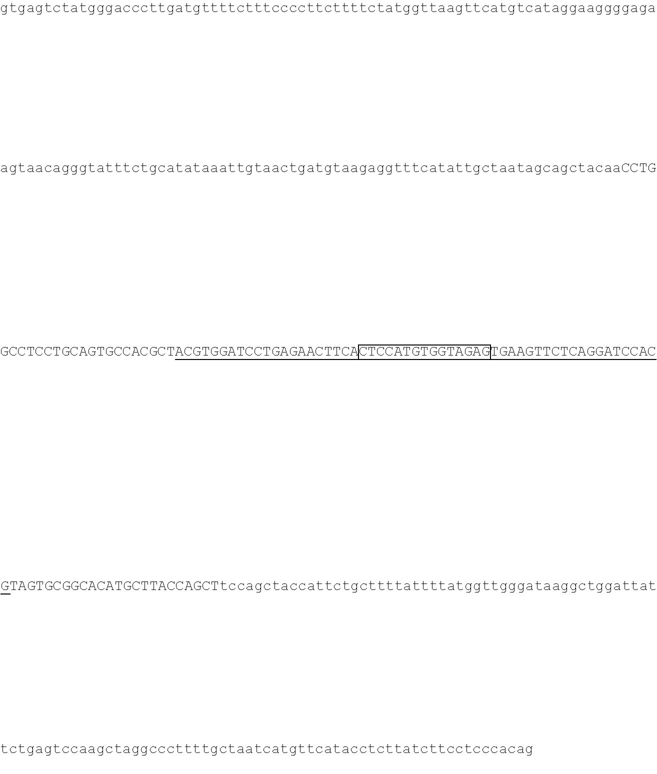

>SEQ ID NO: 80 (βAS3-int2/miR#9)

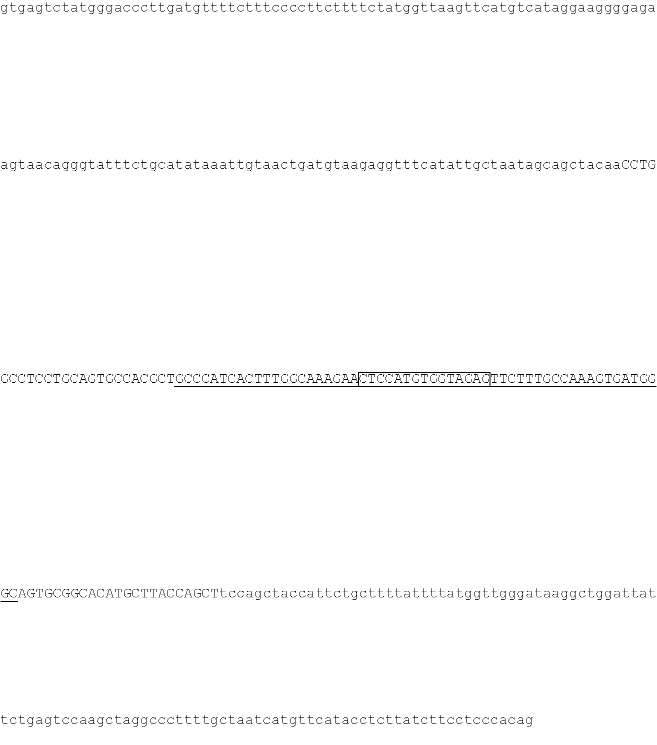

>SEQ ID NO: 81 (βAS3-int2/miR#10)

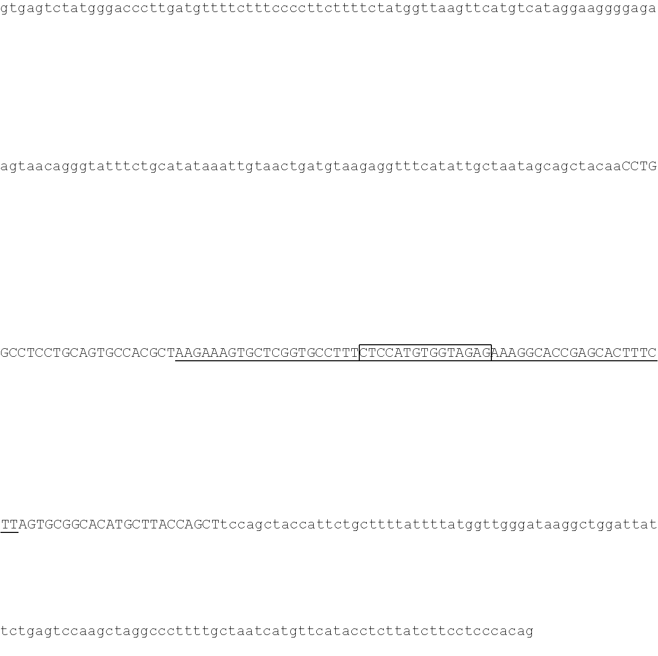

>SEQ ID NO: 82 (βAS3-int2/miR#11)

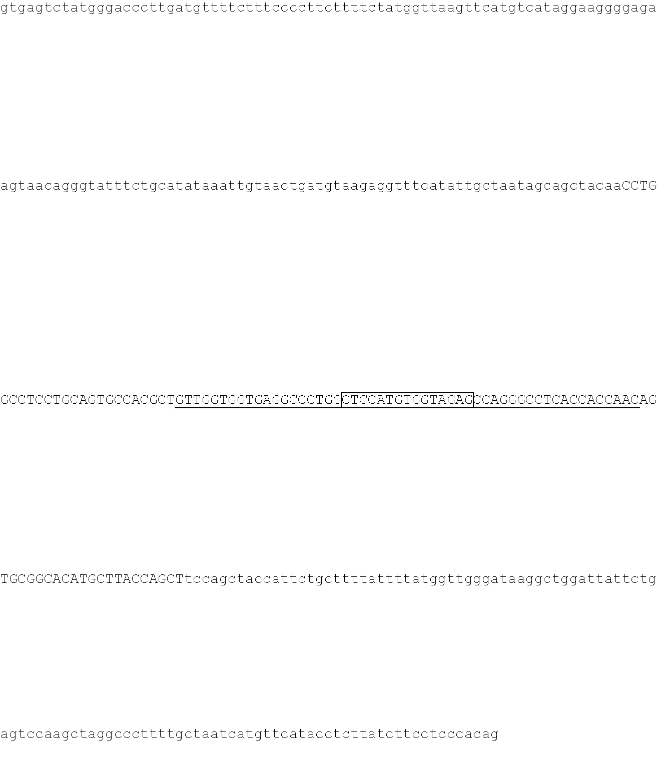

>SEQ ID NO: 83 (βAS3-int2/miR#12)

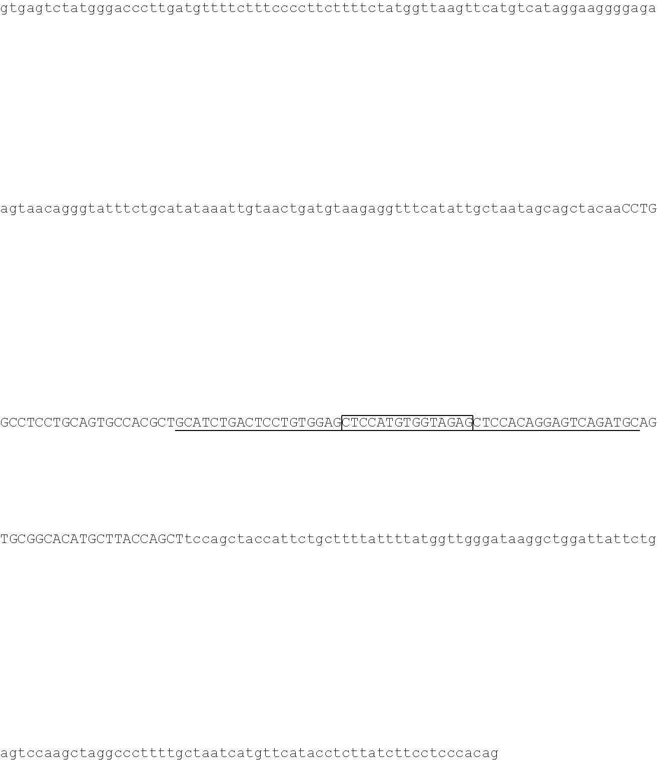

>SEQ ID NO: 84 (βAS3-int2/miR#12mod)

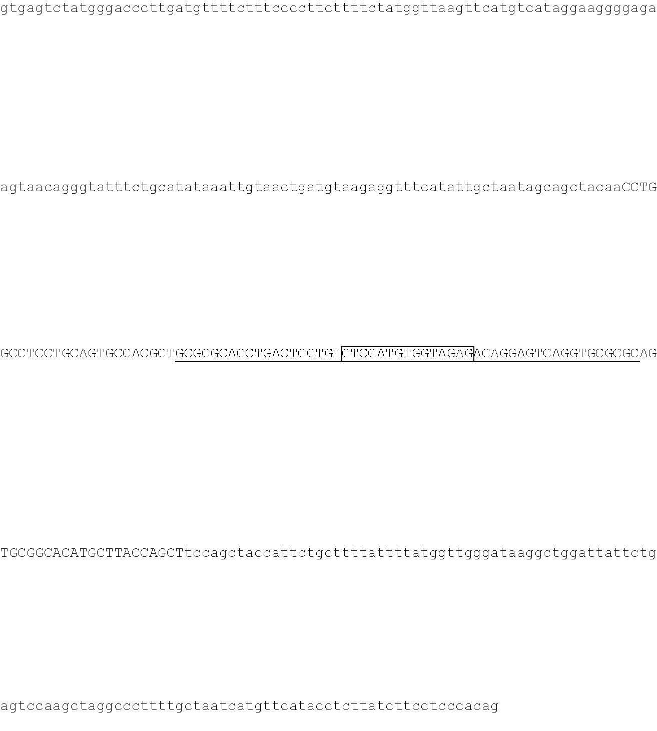

>SEQ ID NO: 85 (βAS3-int2/miR#13)

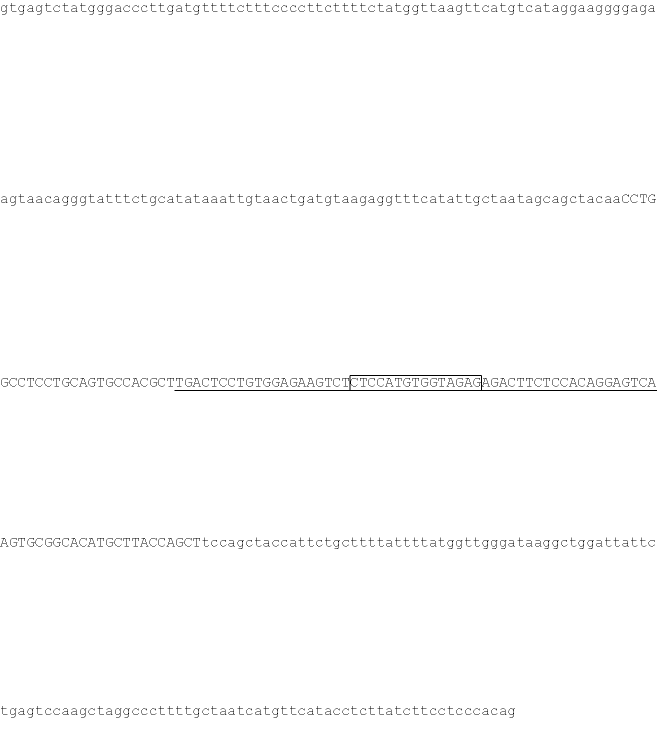

A further object of the present invention relates to a transgene encoding for an anti-sickling HBB, wherein said transgene comprises the nucleic acid molecule of the present invention.

As used herein, the term “anti-sickling HBB”, “AS3’ or “βAS3” refers to a HBB polypeptide that contains three mutations causing three potentially beneficial “anti-sickling” amino-acidic substitutions G16D, E22A, T87Q. Mutation E22A and T87Q impair, respectively, the axial and lateral contacts necessary for the formation of HbS polymers, and mutation G16D increases the affinity to HBA chains, thus conferring to βAS3 a competitive advantage for the incorporation in the Hb tetramers.

In some embodiments, the transgene of the present invention encodes for the βAS3 polypeptide that consists of the amino acid sequence as set forth in SEQ ID NO:2 wherein the amino acid residue G at position 16 is substituted by the amino acid residue D, the amino acid residue E at position 22 is substituted by the amino acid residue A, and the amino acid residue T at position 87 is substituted by the amino acid residue Q.

As used herein, the term “transgene” refers to any nucleic acid that shall be expressed in a mammal cell.

In some embodiments, the transgene of the present invention relates to the transgene described in Weber, L., et al. “An optimized lentiviral vector efficiently corrects the human sickle cell disease phenotype.” Molecular Therapy-Methods & Clinical Development 10 (2018): 268-280, wherein intron 2 sequence is substituted by the nucleic acid molecule of the present invention (e.g. SEQ ID NO:66-85).

In some embodiments, the transgene comprises a least one silent mutation so that the expression of the βAS3 polypeptide will not be not reduced or silenced by amiR of the present invention when the transgene will be expressed. Thus in some embodiments, the transgene comprises in the region encoding for the βAS3 polypeptide at least one silent mutation to impair the binding of the amiR.

As used herein, the term “silent mutation” has its general meaning in the art and means that the one or more DNA mutations that are introduced into the nucleic acid construct do not result in a change to the amino acid sequence of the encoded protein. The silent mutations may occur in a non-coding region (ie. outside of a gene or within an intron). Typically, the silent mutation(s) will occur within an exon in a manner that does not alter the final amino acid sequence of the βAS3 polypeptide. The term “silent mutation” is used interchangeably with the term “synonymous mutation”, however, synonymous mutations are a subcategory of a silent mutation and refer to silent mutations occurring only within exons.

In some embodiments, the transgene comprises the sequence as set forth in SEQ ID NO:86 or SEQ ID NO:87 wherein the 5′ to 3′ sequence of the βAS3 transgene are in lowercase, the amiR sequence is in uppercase, the sequence of shRNA is underlined and the loop of the amiR is framed, and the region which could be targeted by the amiR and that was modified to impair miR binding is double underlined (nucleotides changes are in upper case).

SEQ ID NO: 86 >modified βAS3 sequence (5′-3′) + (βAS3-int2/miR#7mod):
acatttgcttctgacacaactgtgttcactagcaacctcaaacagacaccatggtgcacctgactcctgaggaga
agtctgccgttactgccctgtgggacaaggtgaacgtggatgccgttggtggtgaggccctgggcaggttggtat
caaggttacaagacaggtttaaggagaccaatagaaactgggcatgtggagacagagaagactcttgggtttctg
ataggcactgactctctctgcctattggtctattttcccacccttaggctgctggtggtctacccttggacccag

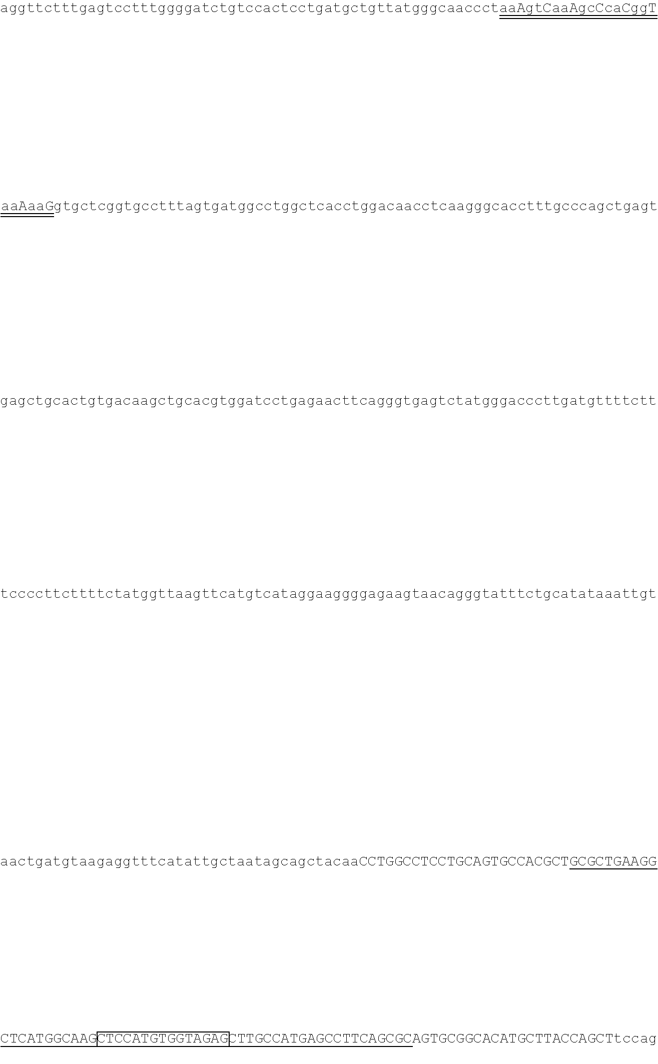

ctaccattctgcttttattttatggttgggataaggctggattattctgagtccaagctaggcccttttgctaat
catgttcatacctcttatcttcctcccacagctcctgggcaacgtgctggtctgtgtgctggcccatcactttgg
caaagaattcaccccaccagtgcaggctgcctatcagaaagtggtggctggtgtggctaatgccctggcccacaa
gtatcactaagctcgctttcttgctgtccaatttctattaaaggttcctttgttccctaagtccaactactaaac
tgggggatattatgaagggccttgagcatctggattctgcctaataaaaaacatttattttcattgcaatgatgt
atttaaattatttctgaatattttactaaaaagggaatgtgggaggtcagtgcatttaaaacataaagaaatgaa
gagctagttcaaaccttgggaaaatacactatatcttaaactccatgaaagaaggtgaggctgcaaacagctaat
gcacattggcaacagcccctgatgcctatgccttattcatccctcagaaaaggattcaagtagaggcttgatttg
gaggttaaagttttgctatgctgtattttacattacttattgttttagctgtcctcatggtacgtaccgataaaa
ttttgaattttgtaatttgtttttgtaattctttagtttgtatgtctgttgctattatgtctactattctttccc
ctgcactgtaccccccaatccccccttttcttttaaaagttaaccgataccgtcgagatccgttcactaatcgaa
tggatctgtctctgtctctctctccaccttcttcttctattccttcgggcctgtcgggtcccctcggggttggga
ggtgggtctgaaacgataatggtgaatatccctgcctaactctattcactatagaaagtacagcaaaaactattc
ttaaacctaccaagcctcctactatcattatgaataattttatataccacagccaatttgttatgttaaaccaat
tccacaaacttgcccatttatctaattccaataattcttgttcattcttttcttgctggttttgcgattcttcaa
ttaaggagtgtattaagcttgtgtaattgttaatttctctgtcccactccatccaggtcgtgtgattccaaatct
gttccagagatttattactccaactagcattccaaggcacagcagtggtgcaaatgagttttccagagcaacccc
aaatccccaggagctgttgatccttt
SEQ ID NO: 87 >modified βAS3 sequence (5′-3′) + (βAS3-int2/miR#10):
acatttgcttctgacacaactgtgttcactagcaacctcaaacagacaccatggtgcacctgactcctgaggaga
agtctgccgttactgccctgtgggacaaggtgaacgtggatgccgttggtggtgaggccctgggcaggttggtat
caaggttacaagacaggtttaaggagaccaatagaaactgggcatgtggagacagagaagactcttgggtttctg
ataggcactgactctctctgcctattggtctattttcccacccttaggctgctggtggtctacccttggacccag
aggttctttgagtcctttggggatctgtccactcctgatgctgttatgggcaaccctaaggtgaaggctcatggc

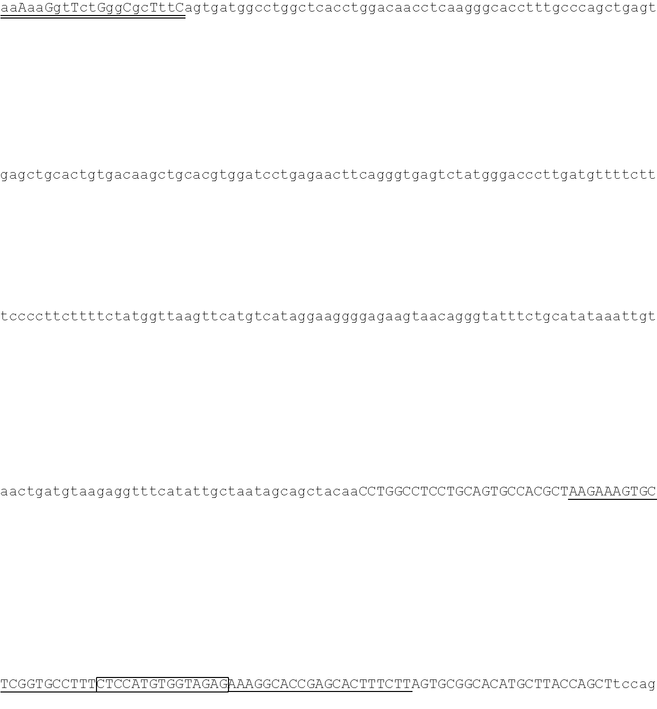

ctaccattctgcttttattttatggttgggataaggctggattattctgagtccaagctaggcccttttgctaat
catgttcatacctcttatcttcctcccacagctcctgggcaacgtgctggtctgtgtgctggcccatcactttgg
caaagaattcaccccaccagtgcaggctgcctatcagaaagtggtggctggtgtggctaatgccctggcccacaa
gtatcactaagctcgctttcttgctgtccaatttctattaaaggttcctttgttccctaagtccaactactaaac
tgggggatattatgaagggccttgagcatctggattctgcctaataaaaaacatttattttcattgcaatgatgt
atttaaattatttctgaatattttactaaaaagggaatgtgggaggtcagtgcatttaaaacataaagaaatgaa
gagctagttcaaaccttgggaaaatacactatatcttaaactccatgaaagaaggtgaggctgcaaacagctaat
gcacattggcaacagcccctgatgcctatgccttattcatccctcagaaaaggattcaagtagaggcttgatttg
gaggttaaagttttgctatgctgtattttacattacttattgttttagctgtcctcatggtacgtaccgataaaa
ttttgaattttgtaatttgtttttgtaattctttagtttgtatgtctgttgctattatgtctactattctttccc
ctgcactgtaccccccaatccccccttttcttttaaaagttaaccgataccgtcgagatccgttcactaatcgaa
tggatctgtctctgtctctctctccaccttcttcttctattccttcgggcctgtcgggtcccctcggggttggga
ggtgggtctgaaacgataatggtgaatatccctgcctaactctattcactatagaaagtacagcaaaaactattc
ttaaacctaccaagcctcctactatcattatgaataattttatataccacagccaatttgttatgttaaaccaat
tccacaaacttgcccatttatctaattccaataattcttgttcattcttttcttgctggttttgcgattcttcaa
ttaaggagtgtattaagcttgtgtaattgttaatttctctgtcccactccatccaggtcgtgtgattccaaatct
gttccagagatttattactccaactagcattccaaggcacagcagtggtgcaaatgagttttccagagcaacccc
aaatccccaggagctgttgatccttt

In some embodiments, the transgene of the present invention is under the transcriptional control of a promoter.

As used herein, the terms “promoter” has its general meaning in the art and refers to a segment of a nucleic acid sequence, typically but not limited to DNA that controls the transcription of the nucleic acid sequence to which it is operatively linked. The promoter region includes specific sequences that are sufficient for RNA polymerase recognition, binding and transcription initiation. In addition, the promoter region can optionally include sequences which modulate this recognition, binding and transcription initiation activity of RNA polymerase. The skilled person will be aware that promoters are built from stretches of nucleic acid sequences and often comprise elements or functional units in those stretches of nucleic acid sequences, such as a transcription start site, a binding site for RNA polymerase, general transcription factor binding sites, such as a TATA box, specific transcription factor binding sites, and the like. Further regulatory sequences may be present as well, such as enhancers, and sometimes introns at the end of a promoter sequence.

As used herein, the terms “operably linked”, or “operatively linked” are used interchangeably herein, and refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

In some embodiments, the transgene of the present invention is placed under the transcriptional control of the HBB promoter and key regulatory elements from the 16-kb human β-locus control region (βLCR), which is essential for high and regulated expression of the endogenous HBB gene family. In some embodiments, the key regulatory elements consist of the 2 DNase I hypersensitive sites HS2 and HS3.

In some embodiments, the transgene is operatively linked to further regulatory sequences.

As used herein, the term “regulatory sequence” is used interchangeably with “regulatory element” herein and refers to a segment of nucleic acid, typically but not limited to DNA, that modulate the transcription of the nucleic acid sequence to which it is operatively linked, and thus acts as a transcriptional modulator. A regulatory sequence often comprises nucleic acid sequences that are transcription binding domains that are recognized by the nucleic acid-binding domains of transcriptional proteins and/or transcription factors, enhancers or repressors etc.

In some embodiments, the sequence of the transgenes is codon-optimized.

As used herein, the term “codon-optimized” refers to nucleic sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell.

In some embodiments, the transgene is inserted in a viral vector, and in particular in a retroviral vector.

As used herein, the term “viral vector” refer to a virion or virus particle that functions as a nucleic acid delivery vehicle and which comprises a vector genome packaged within the virion or virus particle. As used herein, the term “retroviral vector” refers to a vector containing structural and functional genetic elements that are primarily derived from a retrovirus. In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus). In some embodiments, the retroviral vector of the present invention is a replication deficient retroviral virus particle, which can transfer a foreign imported RNA of a gene instead of the retroviral mRNA.

In some embodiments, the retroviral vector of the present invention is a lentiviral vector.

As used herein, the term “lentiviral vector” refers to a vector containing structural and functional genetic elements that are primarily derived from a lentivirus. In some embodiments, the lentiviral vector of the present invention is selected from the group consisting of HIV-1, HIV-2, SIV, FIV, EIAV, BIV, VISNA and CAEV vectors. In some embodiments, the lentiviral vector is a HIV-1 vector. The structure and composition of the vector genome used to prepare the retroviral vectors of the present invention are in accordance with those described in the art. Especially, minimum retroviral gene delivery vectors can be prepared from a vector genome, which only contains, apart from the nucleic acid molecule of the present invention, the sequences of the retroviral genome which are non-coding regions of said genome, necessary to provide recognition signals for DNA or RNA synthesis and processing. In some embodiment, the retroviral vector genome comprises all the elements necessary for the nucleic import and the correct expression of the polynucleotide of interest (i.e. the transgene). As examples of elements that can be inserted in the retroviral genome of the retroviral vector of the present invention are at least one (preferably two) long terminal repeats (LTR), such as a LTR5′ and a LTR3′, a psi sequence involved in the retroviral genome encapsidation, and optionally at least one DNA flap comprising a cPPT and a CTS domains. In some embodiments of the present invention, the LTR, preferably the LTR3′, is deleted for the promoter and the enhancer of U3 and is replaced by a minimal promoter allowing transcription during vector production while an internal promoter is added to allow expression of the transgene. In particular, the vector is a Self-INactivating (SIN) vector that contains a non-functional or modified 3′ Long Terminal Repeat (LTR) sequence. This sequence is copied to the 5′ end of the vector genome during integration, resulting in the inactivation of promoter activity by both LTRs. Hence, a vector genome may be a replacement vector in which all the viral coding sequences between the 2 long terminal repeats (LTRs) have been replaced by the nucleic acid molecule of the present invention.

In some embodiments, the retroviral vector genome is devoid of functional gag, pol and/or env retroviral genes. By “functional” it is meant a gene that is correctly transcribed, and/or correctly expressed. Thus, the retroviral vector genome of the present invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In some embodiments, the retroviral genome is devoid of gag, pol and/or env retroviral genes.

In some embodiments the retroviral vector genome is also devoid of the coding sequences for Vif-, Vpr-, Vpu- and Nef-accessory genes (for HIV-1 retroviral vectors), or of their complete or functional genes.

In some embodiments, the vector of the present invention comprises a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

In some embodiments, the vector of the present invention comprises a Rev Response Element (RRE) to enhance nuclear export of unspliced RNA. RREs are well known to those of skill in the art. Illustrative RREs include, but are not limited to RREs such as that located at positions 7622-8459 in the HIV NL4-3 genome (Genbank accession number AF003887) as well as RREs from other strains of HIV or other retroviruses.

Typically, the retroviral vector of the present invention is non replicative i.e., the vector and retroviral vector genome are not able to form new particles budding from the infected host cell. This may be achieved by the absence in the retroviral genome of the gag, pol or env genes, as indicated in the above paragraph; this can also be achieved by deleting other viral coding sequence(s) and/or cis-acting genetic elements needed for particles formation.

The retroviral vectors of the present invention can be produced by any well-known method in the art including transient transfection (s) in cell lines. Use of stable cell lines may also be preferred for the production of the vectors. For instance, the retroviral vector of the present invention is obtainable by a transcomplementation system (vector/packaging system) by transfecting in vitro a permissive cell (such as 293T cells) with a plasmid containing the retroviral vector genome of the present invention, and at least one other plasmid providing, in trans, the gag, pol and env sequences encoding the polypeptides GAG, POL and the envelope protein(s), or for a portion of these polypeptides sufficient to enable formation of retroviral particles. As an example, permissive cells are transfected with a) transcomplementation plasmid, lacking packaging signal psi and the plasmid is optionally deleted of accessory genes vif, nef, vpu and/or vpr, b) a second plasmid (envelope expression plasmid or pseudotyping env plasmid) comprising a gene encoding an envelope protein(s) and c) a transfer vector plasmid comprising a recombinant retroviral genome, optionally carrying the deletion of the U3 promoter/enhancer region of the 3′ LTR, including, between the 5′ and 3′ retroviral LTR sequences, a psi encapsidation sequence, a nuclear export element (preferably RRE element of HIV or other retroviruses equivalent), and the nucleic acid molecule of the present invention, and optionally a promoter and/or a sequences involved in the nuclear import (cPPT and CTS) of the RNA. Advantageously, the three plasmids used do not contain homologous sequence sufficient for recombination. Nucleic acids encoding gag, pol and env cDNA can be advantageously prepared according to conventional techniques, from viral gene sequences available in the prior art and databases. The trans-complementation plasmid provides a nucleic acid encoding the proteins retroviral gag and pol. These proteins are derived from a lentivirus, and most preferably, from HIV-1. The plasmid is devoid of encapsidation sequence, sequence coding for an envelope, accessory genes, and advantageously also lacks retroviral LTRs. Therefore, the sequences coding for gag and pol proteins are advantageously placed under the control of a heterologous promoter, e.g. cellular, viral, etc., which can be constitutive or regulated, weak or strong. It is preferably a plasmid containing the transcomplementing sequence Δpsi-CMV-gag-pol-PolyA. This plasmid allows the expression of all the proteins necessary for the formation of empty virions, except the envelope glycoproteins. The transcomplementation plasmid may advantageously comprise the TAT and REV genes. The transcomplementation plasmid is advantageously devoid of vif, vpr, vpu and/or nef accessory genes. It is understood that the gag and pol genes and genes TAT and REV can also be carried by different plasmids, possibly separated. In this case, several transcomplementation plasmids are used, each encoding one or more of said proteins. The promoters used in the transcomplementation plasmid, the envelope plasmid and the transfer vector plasmid respectively to promote the expression of gag and pol, of the coat protein, and the mRNA of the vector genome (including the transgene) are promoters identical or different, chosen advantageously from ubiquitous promoters or cell-specific, for example, the viral CMV, TK, RSV LTR promoters and the RNA polymerase III promoters such as U6 or H1. For the production of the retroviral vector of the present invention, the plasmids described above can be introduced into appropriate cells and viruses produced are harvested. The cells used may be any cell particularly eukaryotic cells, in particular mammalian, e.g. human or animal. They can be somatic or embryonic stem or differentiated cells. Typically the cells include 293T cells, fibroblast cells, hepatocytes, muscle cells (skeletal, cardiac, smooth, blood vessel, etc.), nerve cells (neurons, glial cells, astrocytes) of epithelial cells, renal, ocular etc. It may also include, insect, plant cells, yeast, or prokaryotic cells. It can also be cells transformed by the SV40 T antigen. The genes gag, pol and env encoded in plasmids can be introduced into cells by any method known in the art, suitable for the cell type considered. Usually, the cells and the plasmids are contacted in a suitable device (plate, dish, tube, pouch, etc . . . ), for a period of time sufficient to allow the transfer of the plasmid in the cells. Typically, the plasmid is introduced into the cells by calcium phosphate precipitation, electroporation, or by using one of transfection-facilitating compounds, such as lipids, polymers, liposomes and peptides, etc. The calcium phosphate precipitation is preferred. The cells are cultured in any suitable medium such as RPMI, DMEM, a specific medium devoid of fetal calf serum, etc. After transfection, the retroviral vectors of the present invention may be purified from the supernatant of the cells. Purification of the retroviral vector to enhance the concentration can be accomplished by any suitable method, such as by chromatography techniques (e.g., column or batch chromatography).

The vector of the present invention is particularly suitable for driving the targeted expression of the transgene in a host cell.

Accordingly, a further object of the present invention relates to a method of obtaining a population of host cells transduced with the transgene of the present invention, which comprises the step of transducing a population of host cells in vitro, ex vivo or in vivo with the vector of the present invention.

As used herein, the term “transduction” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transduced”.

In some embodiments, the host cell is selected from the group consisting of hematopoietic stem/progenitor cells, hematopoietic progenitor cells, hematopoietic stem cells (HSCs), pluripotent cells (i.e. embryonic stem cells (ES) and induced pluripotent stem cells (iPS)).

Typically, the host cell results from a stem cell mobilization.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment. As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C—C motif) receptor 1 (CCR1), such as chemokine (C—C motif) ligand 3 (CCL3, also known as macrophage inflammatory protein-1α (Mip-1α));

agonists of the chemokine (C—X—C motif) receptor 1 (CXCR1) and 2 (CXCR2), such as chemokine (C—X—C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1,; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38−CD90+CD45RA−, Lin-CD34+CD38−CD90−CD45RA−, Lin-CD34+CD38+IL-3aloCD45RA−, and Lin-CD34+CD38+CD10+ (Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the host cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

Typically, the host cell is isolated. As used herein, the term “isolated cell” refers to a cell that has been removed from an organism in which it was originally found, or a descendant of such a cell. Optionally the host cell has been cultured in vitro, e.g., in the presence of other cells. Optionally the host cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

Methods for transducing host cells are well known in the art. In some embodiments, the host cells may be cultured in the presence of the retroviral vector for a duration of about 10 minutes to about 72 hours, about 30 minutes to about 72 hours, about 30 minutes to about 48 hours, about 30 minutes to about 24 hours, about 30 minutes to about 12 hours, about 30 minutes to about 8 hours, about 30 minutes to about 6 hours, about 30 minutes to about 4 hours, about 30 minutes to about 2 hours, about 1 hour to about 2 hours, or any intervening period of time. During transduction, the host cells may be cultured in media suitable for the maintenance, growth, or proliferation of the host cells. Suitable culture media and conditions are well known in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of hematopoietic cells SFEM®. Many media are also available as low-glucose formulations, with or without sodium pyruvate. During transduction, the host cells may be cultured under conditions that promote the expansion of stem cells or progenitor cells. Any method known in the art may be used. In some embodiments, during transduction, the host cells are cultured in the presence of one or more growth factors that promote the expansion of stem cells or progenitor cells. Examples of growth factors that promote the expansion of stem cells or progenitor cells include, but are not limited to, fetal liver tyrosine kinase (Flt3) ligand, stem cell factor (SCF), and interleukins 6 and 11, which have been demonstrated to promote self-renewal of murine hematopoietic stem cells. Others include Sonic hedgehog, which induces the proliferation of primitive hematopoietic progenitors by activation of bone morphogenetic protein 4, Wnt3a, which stimulates self-renewal of HSCs, brain derived neurotrophic factor (BDNF), epidermal growth factor (EGF), fibroblast growth factor (FGF), ciliary neurotrophic factor (CNF), transforming growth factor-β (TGF-α), a fibroblast growth factor (FGF, e.g., basic FGF, acidic FGF, FGF-17, FGF-4, FGF-5, FGF-6, FGF-8b, FGF-8c, FGF-9), granulocyte colony stimulating factor (GCSF), a platelet derived growth factor (PDGF, e.g., PDGFAA, PDGFAB, PDGFBB), granulocyte macrophage colony stimulating factor (GMCSF), stromal cell derived factor (SCDF), insulin like growth factor (IGF), thrombopoietin (TPO) or interleukin-3 (IL-3). In some embodiments, before transduction, the host cells are cultured in the presence of one or more growth factors that promote expansion of stem cells or progenitor cells. In some embodiments, transduction efficiency is significantly increased by contacting the host cells with the retroviral vector in presence of one or more compounds that stimulate the prostaglandin EP receptor signaling pathway, selected from the group consisting of: a prostaglandin, PGE2; PGD2; PGI2; Linoleic Acid; 13(s)-HODE; LY171883; Mead Acid; Eicosatrienoic Acid; Epoxyeicosatrienoic Acid; ONO-259; Cay1039; a PGE2 receptor agonist; 16,16-dimethyl PGE2; 19(R)-hydroxy PGE2; 16,16-dimethyl PGE2 p-(p-acetamidobenzamido) phenyl ester; 11-deoxy-16,16-dimethyl PGE2; 9-deoxy-9-methylene-16,16-dimethyl PGE2; 9-deoxy-9-methylene PGE2; Butaprost; Sulprostone; PGE2 serinol amide; PGE2 methyl ester; 16-phenyl tetranor PGE2; 15(S)-15-methyl PGE2; 15(R)-15-methyl PGE2; BIO; 8-bromo-cAMP; Forskolin; Bapta-AM; Fendiline; Nicardipine; Nifedipine; Pimozide; Strophanthidin; Lanatoside; L-Arg; Sodium Nitroprusside; Sodium Vanadate; Bradykinin; Mebeverine; Flurandrenolide; Atenolol; Pindolol; Gaboxadol; Kynurenic Acid; Hydralazine; Thiabendazole; Bicuclline; Vesamicol; Peruvoside; Imipramine; Chlorpropamide; 1,5-Pentamethylenetetrazole; 4-Aminopyridine; Diazoxide; Benfotiamine; 12-Methoxydodecenoic acid; N-Formyl-Met-Leu-Phe; Gallamine; IAA 94; and Chlorotrianisene.

Typically, the host cells can be then delivered to a subject in which the transgene encoding for the anti-sickling β-globin will be expressed concomitantly with the artificial miRNA of the present invention that will silence the expression of the β^{S}-globin.

Thus the host cells of the present invention can particularly be useful for the treatment of sickle cell disease.

Accordingly, a further object of the present invention relates to a method of treating sickle cell disease in a subject in need thereof, the method comprising transplanting a therapeutically effective amount of a population of host cells obtained by the method as above described.

In some embodiments, the population of host cells is autologous to the subject, meaning the population of cells is derived from the same subject.

As used herein, the term “sickle cell disease” or “SCD” has its general meaning in the art and refers to a group of autosomal recessive genetic blood disorders, which results from mutations in a globin gene and which is characterized by red blood cells that assume an abnormal, rigid, sickle shape. They are defined by the presence of βS-globin gene coding for a β-globin chain variant in which glutamic acid is substituted by valine at amino acid position 6 of the peptide: incorporation of the βS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus-thalassaemia (HbS/β+), or sickle beta-zerothalassaemia (HbS/β0).

By a “therapeutically effective amount” is meant a sufficient amount of population of host cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the host cells are formulated by first harvesting them from their culture medium, and then washing and concentrating the host cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the host cells with the desired specificity, on the age and weight of the recipient, and on the severity of the targeted condition. This amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 million of cells per kg. Usually 2 to 20 million of cells are injected in the subject. The desired purity can be achieved by introducing a sorting step. For uses provided herein, the host cells are generally in a volume of a liter or less, can be 500 ml or less, even 250 ml or 100 ml or less. The clinically relevant number of cells can be apportioned into multiple infusions that cumulatively equal or exceed the desired total amount of cells.

The invention will be further illustrated by the following figures and examples. However, these examples and figures should not be interpreted in any way as limiting the scope of the present invention.

### FIGURES

FIG. 1: Design of a new LV expressing the β^{AS3}-globin and a miR targeting the β^{S}-globin. (A) Structure of the LV-AS3 vector. (B) Structure of the newly generated LV-AS3/miR #HBB. In (B) the sequence of the miR #HBB is detailed. The sequence highlighted in grey corresponds to the guide strand of the amiR. (C) Schematic view of the miR #HBB target regions within the HBB mRNA. (D) Alignments of miR #7mod (in bold) and miR #10 (underlined) guide strands on HBB exon 2 (reverse complement). Δ, deleted HIV-1 U3 region; SD and SA, HIV splicing donor and acceptor sites; Ψ, HIV-1 packaging signal; RRE, HIV-1 Rev responsive element; Ex, exons of the human HBB; β-p, promoter of HBB; HS2, 3, DNase I hypersensitive site 2, and 3 of human HBB LCR; arrows indicate the mutations introduced in exon 1 (generating amino acid substitutions G16D and E22A) and exon 2 (generating amino acid substitution T87Q).

FIG. 2: Screening of 17 LV-AS3/miR #HBBs in K562 cells. K562 cells were transduced with control (Ctrl; pool of LV-AS3 and -AS3/miR #nt) or miR #HBB-containing LVs (AS3/miR #HBB, miRs from #1 to #11 and their modified [mod] version for miRs #1 and #3 to 7). β^{AS3} mRNA expression was measured by RT-qPCR in K562 cells and normalized to HBA (α-globin). The levels of β^{AS3} relative expression per VCN are shown in this histogram (mean +/−SD). The control corresponds to the mean of the results obtained with AS3 and AS3/miR #nt lentiviral vectors and was set to 100%.

FIG. 3: miR-mediated β- and β^{AS3}-globin down-regulation. (A) β^{AS3}-globin mRNA expression normalized to HBA (α-globin) per VCN. (B) β-globin mRNA expression normalized to HBA. (C-D) Western Blot analysis of (β+β^{AS3}) globins chains (C) and quantification relative to α-globin (D) Analyses were performed in mature erythroblasts derived from HUDEP-2 cells transduced with either LV-AS3/miR #HBB or LV-AS3/miR #nt control vector at a high MOI of 10 or a low MOI of 2. Results are represented as % of the control and shown as mean +/−SD. VCN is indicated below each graphs.

FIG. 4: miR-mediated β^{S}-globin down-regulation in primary BFU-E from SCD patients. (A) Frequency of BFU-E and CFU-GM in mock- and LV-transduced HSPCs. Results are represented as % of colonies obtained from 500 plated HSPCs and shown as mean +/−SD. (B) β^{AS3}-globin and β-globin (HBB) mRNA expression normalized to HBA (α-globin). (n=6 controls, Ctrl). (C) Analysis of HbS, HbAS3 and HbF by CE-HPLC. We calculated the percentage of each Hb type over the total Hb tetramers (n=2). HSPCs were transduced using a MOI of 2 or 50. VCN in BFU-E is indicated below each graph.

FIG. 5: miR #7mod-mediated β^{S}-globin down-regulation in erythroid precursors from SCD patients. Data were obtained in erythroid precursors differentiated from SCD HSPCs (3 donors) that were either mock-transduced or transduced with either LV-AS3mod/miR #7mod (AS3modmiR #7mod) or control LVs (Ctrl, LV-AS3, LV-AS3mod, or LV-AS3mod/miR #nt) vectors. VCN is indicated below each graph. (A) β^{S}-globin mRNA expression normalized to HBA (α-globin). ****P<0.0001, Mann-Whitney test. (B) β^{AS3}-globin mRNA expression normalized to HBA (α-globin). ns, Mann-Whitney test.

FIG. 6: LV-AS3mod/miR #7mod decreases HbS levels and improves the SCD cell phenotype in mature RBCs from SCD patients. Data were obtained in RBCs differentiated from SCD HSPCs (2 donors) that were either mock-transduced or transduced with either LV-AS3mod/miR #7mod (AS3modmiR #7mod) or with control LVs (Ctrl, LV-AS3, LV-AS3mod, or LV-AS3mod/miR #nt) vectors. VCN is indicated below each graph. (A-B) HbS and HbAS3 expression in mature RBCs measured by CE-HPLC. **P<0.01, Mann-Whitney test. (C) Proportion of HbS-positive RBCs measured by flow cytometry analysis using an antibody recognizing specifically HbS. ***P<0.001, Mann-Whitney test. (D) Frequency of sickling RBCs after 1-hour incubation at low oxygen tension (0% O2). **P<0.01, Mann-Whitney test.

FIG. 7: Enucleation and RBC differentiation were not altered upon β^{S}-globin silencing induced by LV-AS3mod/miR #7mod. Data were obtained in RBCs differentiated (day 6, 13 or 20 of differentiation) from SCD HSPCs (2 donors) that were either mock-transduced (Mock) or transduced with either LV-AS3mod/miR #7mod (AS3modmiR #7mod, VCN=1.9±0.6) or with control LVs (Ctrl, LV-AS3, LV-AS3mod, or LV-AS3mod/miR#nt, VCN=2.0±0.7) vectors. (A) Frequency of enucleated RBCs measured by flow cytometry at day 13 and 20 of erythroid differentiation. (B) Frequencies of (right) CD36⁺, (middle) CD71⁺, (left) and CD235a⁺ RBCs measured by flow cytometry along the differentiation (day 6, 13 and 20). During erythroid differentiation, cells progressively lose CD36 and CD71 expression (C) Frequencies of CD49⁺, Band3⁺ and CD49⁺Band3⁺ cells among the CD235a⁺ RBCs measured by flow cytometry along the differentiation (day 6, 13 and 20). During erythroid differentiation, CD235a⁺ cells lose progressively lose the CD49 marker and express band 3.

### EXAMPLE

### Material & Methods

### Molecular Cloning

We digested PUC-57 plasmids containing our inserts of interest (AS3mod/miR#7mod, AS3mod/miR #10, AS3mod/miR #nt, AS3mod) and the target plasmid allowing LV production (P_GLOBE) using SwaI and ClaI restrictions enzymes (NEB) at 37° C. overnight. SwaI and ClaI were heat inactivated at 65° C. for 20 min. Digested plasmids were then loaded on a 1% agarose gel and migrated 1 h at 100 V. The fragments of interest were then extracted from the gel and purified with the gel purification Qiagen quick kit. P_GLOBE fragments were dephosphorylated with alkaline phosphatase 1 h at 37° C. and the reaction was stopped by adding EGTA at a final concentration of 18 mM. We then ligated the inserts in the P_GLOBE plasmid using T4 ligase at room temperature for 15 min. To perform the ligation step, we used a 1/6 vector/insert ratio.

XL-10 Gold Ultracompetent Cells were transformed with the new constructs according to Stratagene protocol. Transformed bacteria were then seeded on LB agar-Ampicillin plates and grown at 37° C. overnight. Individual colonies were amplified in LB-Ampicillin overnight at 30° C. under shaking to purify plasmids (PureLink HiPure Miniprep Kit, Invitrogen) and identify the correct plasmid. The selected colonies were amplified in LB-Ampicillin at 30° C. overnight under shaking to purify plasmids (PureLink HiPure Maxiprep Kit, Invitrogen) and produce LV.

### Lentiviral Vector Production and Titration

Third-generation LVs were produced by calcium phosphate transient transfection of HEK293T cells with the transfer vector, the packaging plasmid pHDMH gpm2 (encoding gag/pol), the Rev-encoding plasmid pBA Rev, and the vesicular stomatitis virus glycoprotein G (VSV-G) envelope-encoding plasmid pHDM-G. The viral infectious titer, expressed as transduction units per ml (TU/ml) was measured in HCT116 cells after transduction using 6 vector volumes (5 μl, 1 μl, 0.5 μl and 0.1 μl, 0.05 μl ). Four days after transduction, genomic DNA was extracted and the vector copy number (VCN) per cell was measured by ddPCR. The LV titer was then calculated as follows: Titer (TU/ml)=volume of vector used/(number of cells at infection*VCN).

Vector Copy Number Quantification by ddPCR

Genomic DNA was extracted from HCT116 cells 4 days after transduction, K562 cells, HUDEP-2 cells and BFU-E 14 days after transduction, using the PureLink Genomic DNA Mini Kit (Invitrogen). DNA was digested using DraI restriction enzyme (NEB) at 37° C. for 30 min and then mixed with the ddPCR reaction mix composed of 2× ddPCR SuperMix for probes (no dUTP) (Bio-Rad), forward (for) and reverse (rev) primers (at a final concentration of 900 nM) and probes (at a final concentration of 250 nM). We used probes and primers specific for: (i) albumin (VIC-labeled ALB probe with a QSY quencher, 5′-CCTGTCATGCCCACACAAATCTCTCC-3′ (SEQ ID NO:88) ; FOR ALB primer, 5′-GCTGTCATCTCTTGTGGGCTGT-3′(SEQ ID NO:89); REV ALB primer, 5′-ACTCATGGGAGCTGCTGGTTC-3′SEQ ID NO:90), and for (ii) the LV (FAM-labeled LV probe with a MGB quencher, 5′-CGCACGGCAAGAGGCGAGG-3′(SEQ ID NO:91); FOR LV primer 5′-TCCCCCGCTTAATACTGACG-3′(SEQ ID NO:92); REV LV primer 5′-CAGGACTCGGCTTGCTGAAG-3′ (SEQ ID NO:93)). The albumin gene was chosen as reference locus to calculate the VCN per diploid genome, as it is present in 2 copies per genome in every human cells. Droplets were generated using a QX200 droplet generator (Bio-Rad) with droplet generation oil for probes (Bio-Rad) onto a DG8 cartridge (Bio-Rad) and transferred on a semi-skirted 96 well plate (Eppendorf AG). After sealing with a pierce-able foil heat seal using a PX1 PCR plate sealer (Bio-Rad), the plate was loaded on a SimpliAmp Thermal Cycler (ThermoFisher Scientific) for PCR amplification using the following conditions: 95° C. for 10 min, followed by 40 cycles at 94° C. for 30 sec and 60° C. for 1 min, and by a final step at 98° C. for 10 min. The plate was analyzed using the QX200 droplet reader (Bio-Rad) (channel 1: FAM, channel 2: VIC) and analyzed using the QuantaSoft analysis software (Bio-Rad), which quantifies positive and negative droplets and calculate the starting DNA concentration using a Poisson algorithm. The average VCN per cell were calculated as (LV copies*2)/(albumin copies).

K562 Cell Culture and Transduction K562 were maintained in RPMI 1640 medium (Lonza) containing glutamine and supplemented with 10% fetal bovine serum (Lonza), HEPES (LifeTechnologies), sodium pyruvate (LifeTechnologies) and penicillin and streptomycin (LifeTechnologies). K562 were transduced at a cell concentration of 5×10⁵ cells/ml in the culture medium supplemented with 4 ug/m1 polybrene (Sigma). After 24 h, cells were washed and cultured in fresh culture medium.

### HUDEP-2 Cell Culture, Differentiation and Transduction

HUDEP-2 cells (HUDEP-2) were cultured and differentiated as previously described (Antoniani et al., 2018; Canver et al., 2015; Kurita et al., 2013). HUDEP-2 cells were expanded in a basal medium composed of StemSpan SFEM (Stem Cell Technologies) supplemented with 10⁻⁶ M dexamethasone (Sigma), 100 ng/ml human stem cell factor (hSCF) (Peprotech), 3 IU/m1 erythropoietin (EPO) Eprex (Janssen-Cilag, France), 100 U/ml L-glutamine (Life Technologies), 2 mM penicillin/streptomycin and 1 μg/ml doxycycline (Sigma). HUDEP-2 cells were transduced at a cell concentration of 10⁶ cells/ml in basal medium supplemented with 4 ug/ml protamine sulfate (Choay). After 24 h, cells were washed and cultured in fresh basal medium. Cells were differentiated for 9 days in Iscove's Modified Dulbecco's Medium (IMDM) (Life Technologies) supplemented with 330 μm/ml holo-transferrin (Sigma), 10 μg/ml recombinant human insulin (Sigma), 2 IU/ml heparin (Sigma), 5% human AB serum (Eurobio AbCys), 3 IU/mL erythropoietin, 100 ng/mL human SCF, 1 μg/ml doxycycline, 100 U/ml L-glutamine, and 2 mM penicillin/streptomycin.

### HSPC Purification and Transduction

Human adult HSPCs were obtained from healthy donors (HD). Written informed consent was obtained from all subjects. All experiments were performed in accordance with the Declaration of Helsinki. The study was approved by the regional investigational review board (reference, DC 2014-2272, CPP Ile-de-France II “Hôpital Necker-Enfants malades”). HSPCs were purified by immunomagnetic selection (Miltenyi Biotec) after immunostaining using the CD34 MicroBead Kit (Miltenyi Biotec).

CD34⁺ cells were thawed and cultured for 24 h at a concentration of 10⁶ cells/mL in pre-activation medium composed of X-VIVO 20 supplemented with penicillin/streptomycin (Gibco) and recombinant human cytokines: 300 ng/mL SCF, 300 ng/mL Flt-3L, 100 ng/mL TPO, 20 ng/mL interleukin-3 (IL-3) (Peprotech) and 10 mM SR1 (StemCell). After pre-activation, cells (3.10⁶ cells/mL) were cultured in pre-activation medium supplemented with 10 μM PGE2 (Cayman Chemical) on RetroNectin coated plates (10 μg/cm2, Takara Bio) for at least 2 h. Cells (3.10⁶cells/mL) were then transduced for 24 h on RetroNectin coated plates in the pre-activation medium supplemented with 10 μM PGE2, protamine sulfate (4 μg/mL, Protamine Choay) and Lentiboost (1 mg/ml, SirionBiotech).

### CFC Assay

The number of hematopoietic progenitors was evaluated by clonal colony-forming cell (CFC) assay. HSPCs were plated at a concentration of 5×10² cells/mL in methylcellulose-containing medium (GFH4435, Stem Cell Technologies) under conditions supporting erythroid and granulo-monocytic differentiation. BFU-E and CFU-GM colonies were scored after 14 days. BFU-Es and CFU-GMs were randomly picked and collected as bulk populations (containing at least 25 colonies) to evaluate transduction efficiency and globin expression.

In Vitro Erythroid Differentiation

Mature RBCs from mock- and LV-transduced CD34⁺ HSPCs were generated using a three-step protocol (Weber et al., 2018). Briefly, from day 0 to 6, cells were grown in a basal erythroid medium (BEM) supplemented with SCF, IL3, erythropoietin (EPO) (Eprex, Janssen-Cilag) and hydrocortisone (Sigma). From day 6 to 20, they were cultured on a layer of murine stromal MS-5 cells in BEM supplemented with EPO from day 6 to day 9 and without cytokines from day 9 to day 20. From day 13 to 20, human AB serum was added to the BEM.

### RT-qPCR Analysis

RNA was extracted from K562 cells, HUDEP-2 cells after 9 days of differentiation or from primary BFU-E using the RNeasy micro kit (QIAGEN). Reverse transcription of mRNA was performed using the SuperScript III First-Strand Synthesis System for RT-PCR (Invitrogen) with oligo(dT)₂₀ primers. qPCR was performed using the SYBR green detection system (BioRad). We used the following primers: βAS3 FOR, 5′-AAGGGCACCTTTGCCCAG-3′ (SEQ ID NO: 94); βAS3 REV, 5′-GCCACCACTTTCTGATAGGCAG-3′ (SEQ ID NO: 95); HBB FOR, 5′-AAGGGCACCTTTGCCACA-3′ (SEQ ID NO: 96); HBB REV, 5′-GCCACCACTTTCTGATAGGCAG-3′ (SEQ ID NO:97); HBA FOR, 5′-CGGTCAACTTCAAGCTCCTAA-3′ (SEQ ID NO:98); HBA REV, 5′-ACAGAAGCCAGGAACTTGTC-3′(SEQ ID NO: 99). The samples were analyzed with the ViiA 7 Real-Time PCR System and software (Applied Biosystems).

### HPLC

Hemoglobin tetramers from BFU-E and RBCs were separated by cation exchange (CE)-HPLC using a 2 cation-exchange column (PolyCAT A, PolyLC, Columbia). Samples were eluted with a gradient mixture of solution A (20 mM bis Tris, 2 mM KCN, pH, 6.5) and solution B (20 mM bis Tris, 2 mM KCN, 250 mM NaCl, pH, 6.8). The absorbance was measured at 415 nm.

### Western Blot

HUDEP-2 cells after 9 days of differentiation or primary BFU-E were lysed for 30 min at 4° C. using a lysis buffer containing: 10 mM Tris, 1 mM EDTA, 0.5 mM EGTA, 1% Triton X-100, 0.1% SDS, 0.1% Na-deoxicholate, 140 mM NaCl (Sigma-Aldrich) and protease inhibitor cocktail (Roche-Diagnostics). Cell lysates were sonicated twice (50% amplitude, 10 sec per cycle, pulse 9 sec on/1 sec off) and underwent 3 cycles of freezing/thawing (3 min at −80° C./3 min at 37° C.). After centrifugation, the supernatant was collected and protein concentration was measured using the Pierce™ BCA Protein Assay Kit (ThermoScientific). After electrophoresis and protein transfer, β- and α-globins were detected using the antibodies sc-21757 and sc-31110 (SantaCruz), respectively. The bands corresponding to β- and α-globins were quantified using the Chemidoc and the Image lab Software (BioRad).

### Flow Cytometry

In primary cell cultures, the expression of erythroid markers was monitored by flow cytometry using anti-CD36, anti-CD49d, anti-CD71, anti-CD235a (BD Horizon) and anti-CD233 (band 3) (IBGRL) antibodies and 7-AAD for cell death assessment. The proportion of enucleated RBCs was measured using the nuclear dye DRAQS (eBioscience). The proportion of HbS-positive RBCs were measured with an antibody recognizing HbS. Briefly, RBCs were stained with a monoclonal mouse anti-human CD235a antibody (BD Biosciences), then fixed 0.05% glutaraldehyde for 10 min at RT, permeabilized with 0.1% Triton X-100 for 10 min at RT and stained with the HbS antibody. Flow cytometry analyses were performed using the Gallios analyzer and Kaluza software (Beckman-Coulter).

### Sickling Assay

At day 19 of the terminal erythroid differentiation, RBCs were collected and incubated in hypoxic conditions to evaluate their sickling properties. Briefly, RBCs were resuspended in ID-CellStab stabilization solution for red cells (BIORAD) and gradually exposed to an oxygen-deprived atmosphere: 10% O2 for 20 min, 5% O2 for 20 min, 0% O2 until control cells were all sickled (between 20 and 80 min). Images were captured using the Axio Observer microscope and the Zen software (Zeiss) at a magnification of 40× at 20% O2 and at each time point under hypoxia (10%, 5%, 0% at 20, 40, 60 and 80 min). Images were analyzed with ImageJ to determine the percentage of sickled RBCs per field of acquisition in the total RBC population.

### Results

### A New LV-Based Strategy to Treat SCD

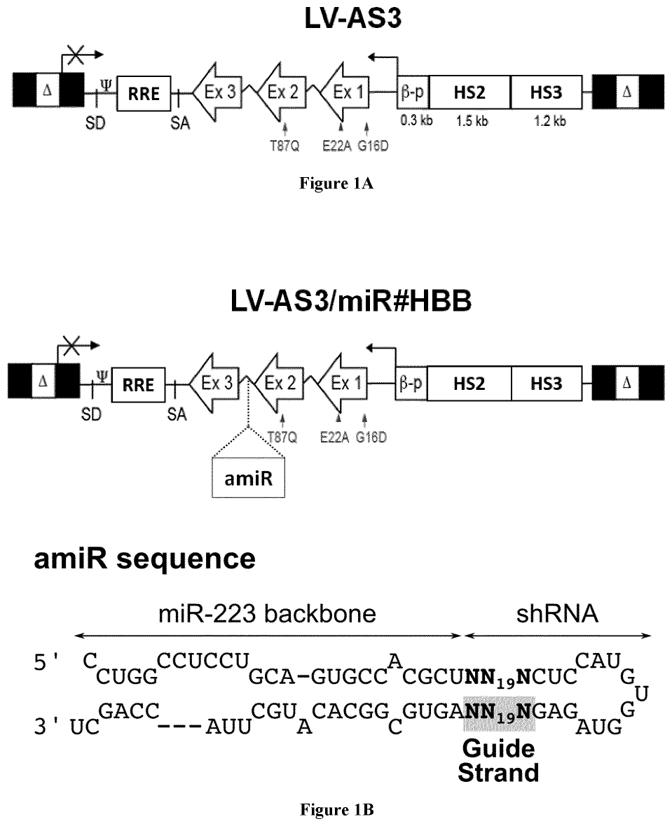

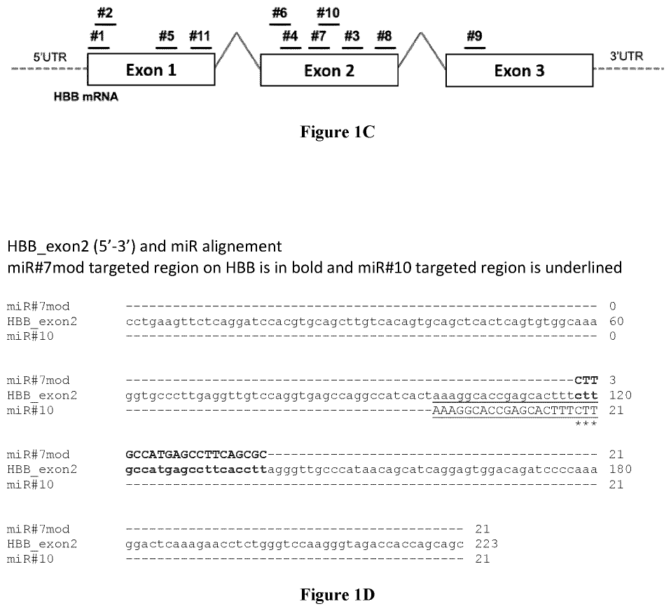

Strategies based on gene addition of therapeutic β-like globin using a LV showed good results in SCD patients who received a drug product with a VCN>1 and when the VCN in vivo was around 2. Unfortunately obtaining a VCN>1 is not always achievable even with optimized protocols and poses some concerns on the low but still possible genotoxicity due to the integration of a high number of vector copies in hematopoietic cells. In addition, Weber et al showed that despite a high transduction efficiency obtained with the LV-AS3 vector (FIG. 1A) the SCD RBC phenotype derived is only partially corrected in in vitro sickling assay. This is probably due to HbS levels that remain high in these cells. Therefore, gene addition strategies using a LV still require improvements to fully correct the SCD phenotype and to show a benefit in patients for which a VCN>1 cannot be obtained.

Here, we have improved the design of the LV-AS3 vector to treat SCD patients. We developed new LVs combining two strategies: gene addition and silencing. These LVs allow the simultaneous expression of the potent anti-sickling β^{AS3}-globin and an artificial miR (amiR) silencing the β^{S}-globin (FIG. 1B). The reduction of β^{S}-globin levels will increase the incorporation of β^{AS3}-globin in Hb tetramers, which should increase the proportion of corrected RBCs in SCD patients. We opted for a miR-based strategy instead of other RNA interference system such as short hairpin RNAs (shRNAs) to reduce the potential toxicity. shRNAs mimic the structure of the pre-miR and can be functional in human cells but they might interfere with the miR processing machinery within the cell due to their high expression levels. miR-based gene therapies are considered safer as miRs are naturally present in human cells. We selected the best-performing miRs, and modified the therapeutic β^{AS3}-globin transgene by inserting silent mutations to avoid the recognition by the amiR and the silencing of the transgene.

As there is no miR targeting the β^{S}-globin available in the literature, we adapted sequences from siRNAs and shRNAs targeting the β^{S}-globin already published or newly designed using a software developed by Adams et al (Adams et al., 2017). These RNA interference sequences were adapted and inserted in the pri-miR-223 backbone to create an amiR (Amendola et al., 2009; Brendel et al., 2020; Guda et al., 2015) (FIG. 1B). We choose the miR-223 backbone because it is a hematopoietic-specific miR and has been improved to allow robust and efficient miR processing. In the final stage of miR maturation, the miR-223 backbone favors the incorporation of the guide strand (the strand that recognize the target mRNA) within the RISC complex over the passenger strand, thus increasing the silencing of the target gene. Indeed, Amendola et al., in their study, developed a LV platform able to deliver amiR derived from the miR-223. In particular, they inserted one or more amiR within an intron of a transgene and obtained a significant silencing of different target genes in several cell types including human primary cells (Amendola et al., 2009). Based on this study and on results obtained in the lab, the different amiRs targeting the β^{S}-globin (miR #HBB) have been inserted within the intron 2 of the transgene. Therefore, the β^{AS3} transgene and the miR #HBB are co-expressed under the control of the β-globin LCR/promoter limiting their expression to the erythroid lineage, thus avoiding potential toxicity in other cell types (FIGS. 1B and 1C). Moreover, the miR #HBBs generated from shRNA sequences have been modified by removing 4 nucleotides at the 5′ end of the guide strand and adding a GCGC (SEQ ID NO:86) motif at the 3′ end. These modifications have been shown to further enhance the selection of the guide strand over the passenger strand in the RISC complex for thermodynamic reasons and therefore would potentially increase β^{S}-globin silencing (Guda et al., 2015). Therefore, we designed 2 versions of the miR #HBBs adapted from shRNAs: the modified (miR #HBBmod, N15-17-GCGC (SEQ ID NO:100)) and the unmodified (miR #HBB, NNNN-N15-17) miRs (Table 1).

TABLE 1
miR sequences targeting the HBB gene

| | | miR sequences (5′-3′) | |
|---|---|---|---|
| ID | Origine | Guide strand | miR types |
| miR#1 | Samakoglu et al., 2006 | CTCCTCAGGAGTCAGATGC | shRNA |
| miR#1 mod | | TCAGGAGTCAGGTGCGCGC | |
| miR#2 | Dykxhoorn et al., 2006 | AGACTTCTCCTCAGGAGTCA | SiRNA |
| miR#3 | portals.broadinstitute.org | TCAGTGTGGCAAAGGTGCCCT | shRNA |
| miR#3mod | | TGTGGCAAAGGTGCCCTGCGC | |
| miR#4 | portals.broadinstitute.org | ATAACAGCATCAGGAGTGGAC | shRNA |
| miR#4mod | | CAGCATCAGGAGTGGACGCGC | |
| miR#5 | portals.broadinstitute.org | TTCATCCACGTTCACCTTGCC | shRNA |
| miR#5mod | | TCCACGTTCACCTTGCCGCGC | |
| miR#6 | portals.broadinstitute.org | CAAAGAACCTCTGGGTCCAAG | shRNA |
| miR#6mod | | GAACCTCTGGGTCCAAGGCGC | |
| miR#7 | portals.broadinstitute.org | CTTTCTTGCCATGAGCCTTCA | shRNA |
| miR#7mod | | CTTGCCATGAGCCTTCAGCGC | |
| miR#8 | Adams et al., 2017 website | TGAAGTTCTCAGGATCCACGT | miR |
| miR#9 | Adams et al., 2017 website | TTCTTTGCCAAAGTGATGGGC | miR |
| miR#10 | Thermofisher | AAAGGCACCGAGCACTTTCTT | SiRNA |
| miR#11 | Dykxhoorn et al., 2006 | CCAGGGCCTCACCACCAAC | SiRNA |
| miR#12 | Samakoglu et al., 2006 | CTCCACAGGAGTCAGATGC | shRNA |
| miR#12mod | | ACAGGAGTCAGGTGCGCGC | |
| miR#13 | Dykxhoorn et al., 2006 | AGACTTCTCCACAGGAGTCA | SiRNA |

GCGC: motif added in miRmod to improve the knock-down
CC: motif added to improve knock-down efficiency of the miR
in bold: matching sequence between the original miR (miR_xx) and the modified version (miR_xxmod)
in italic: the miR is modified to target the βS-mRNA only

miR#HBB Screening in K562 Cells

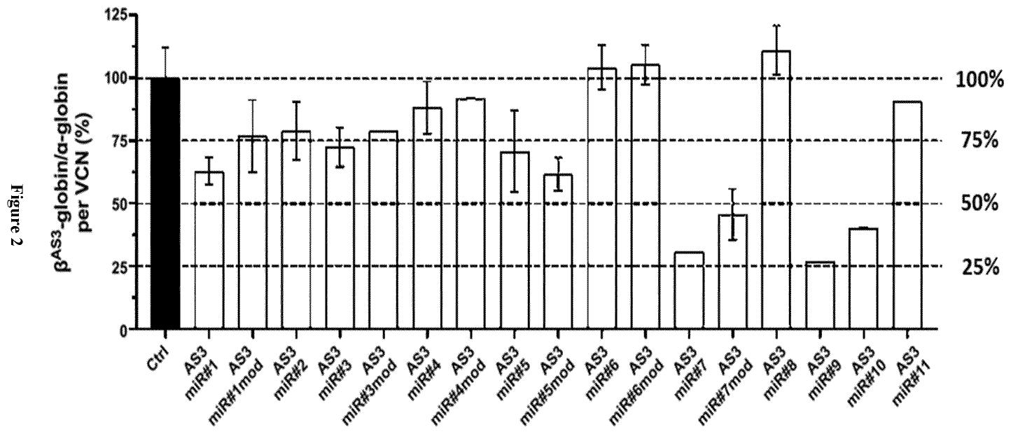

We have generated 17 LVs (LVs-AS3/miR #HBB) co-expressing the potent anti-sickling β^{AS3}-globin and each of the 17 newly designed miR #HBBs (miR #1 to #11, miR #lmod and miR#3mod to #7mod). In order to determine the best performing amiR, we transduced K562 erythroleukemic cells (that do not express endogenous β-globin) with the 17 LVs-AS3/miR #HBB using a multiplicity of infection (MOI) of 15 and 3. For this amiR screening, the β^{AS3} transgene sequence was not modified and could be targeted by the 17 different amiRs. Indeed, there were no mismatches between the miR#HBBs and their target sequences within the β^{AS3} transgene. Therefore, we assessed the silencing effect of the amiRs on the β^{AS3} transgene. The ratio between the amiR and the β^{AS3} mRNA should be independent from the VCN as they derive from the same primary transcript. Similarly, the percentage decrease of the β^{AS3} expression upon miR-mediated silencing should be stable regardless the VCN. Therefore, in this experiment, we compared β^{AS3} expression normalized per VCN in cells transduced with LVs-AS3/miR #HBB to cells transduced with control LVs, which either express the β^{AS3}-globin alone (LV-AS3) or co-express the β^{AS3}-globin with a non-targeting (nt) amiR that does not recognize any human sequence (LV-AS3/miR #nt). K562 cells transduced with AS3/miR #7, #7mod, #9 and ∩10 LVs showed a decrease of more than 50% in β^{AS3} mRNA expression compared to cells transduced with control LVs (FIG. 2). However, the cells transduced with LV-AS3/miR #9 have a very low VCN (<0.5) in comparison with other LVs, which could reflect a low gene transfer efficiency that hampers its use as a therapeutic vector for SCD. In cells transduced with LVs AS3/miR #1 and #5mod, we observed a reduction in β^{AS3} expression of around 30% compared to control cells (FIG. 2). Based on these results we decided to select miR #1, #5mod, miR #7, #7mod and #10 to further characterize these miR #HBBs in HUDEP-2 cells.

Validation of efficient LV-AS3/miR#HBBs in HUDEP-2 cells

In K562 cells, 5 efficient LV-AS3/miR #HBBs (miR #1, #5mod, #7, #7mod and #10) were able to downregulate the expression of the β^{AS3} transgene. To confirm that these LVs downregulate the endogenous β-globin gene, we tested the LV-AS3/miR #HBB expressing the selected miR #HBBs in HUDEP-2, an erythroid progenitor cell line. These cells can be differentiated to mature erythroid precursors expressing the endogenous α and β-globin chains at both mRNA and protein levels. Contrary to the β^{AS3} transgene for which the expression and the miR-mediated silencing are VCN-independent (as discussed for the experiment in K562 cells), the endogenous β-globin and miR #HBB are not expressed at the same molar ratio. Indeed, the decrease of HBB expression should be VCN-dependent, and, more precisely, proportional to the VCN.

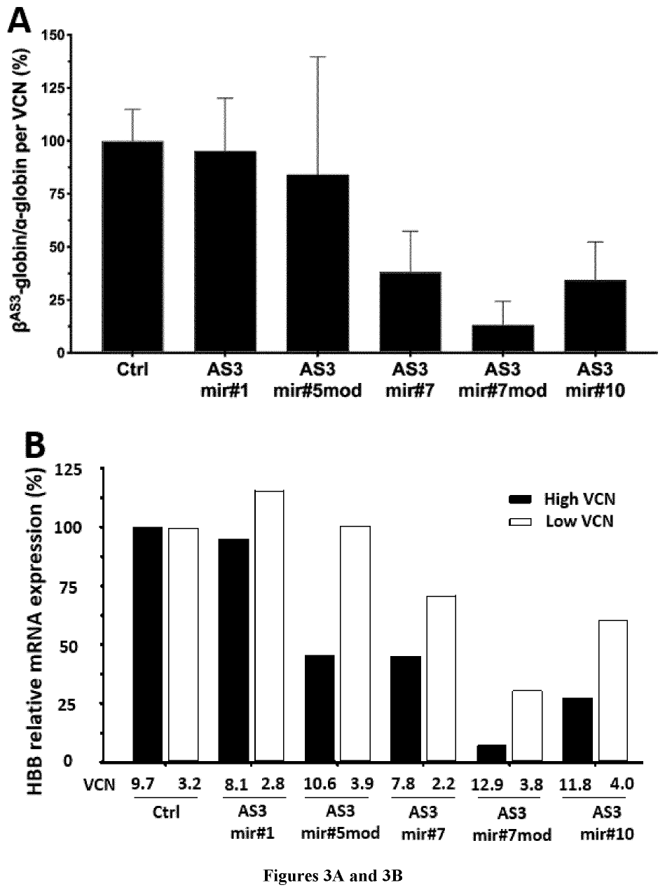

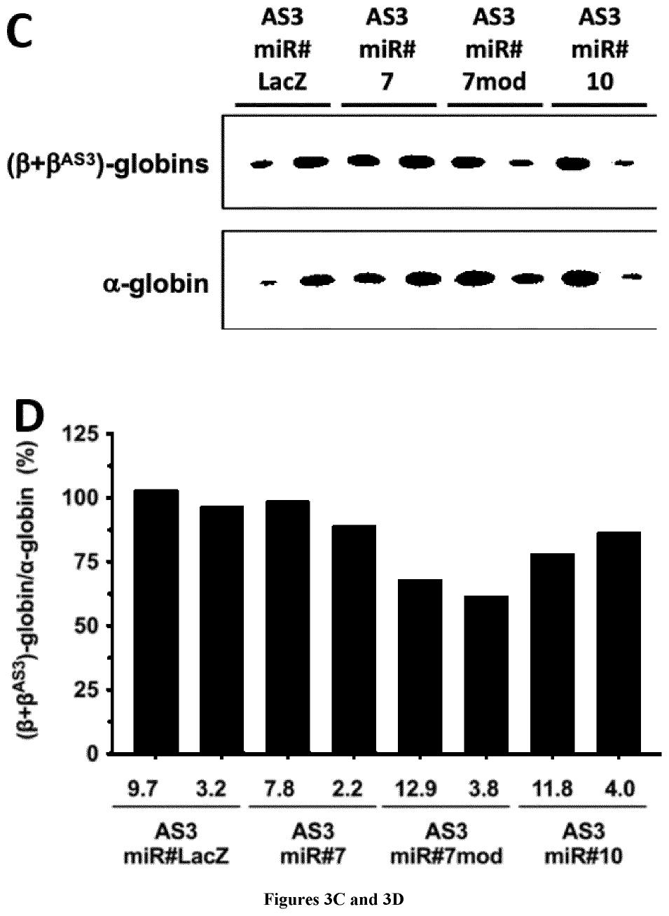

HUDEP-2 cells were transduced with control (AS3/miR #nt) and miR #HBB LVs at a high MOI of 10 or a low MOI of 2 and differentiated to evaluate miR efficiency in down-regulating HBB and β^{AS3} expression. We observed a strong reduction of the β^{AS3} transcripts per VCN (from ˜60% to 85%) in cells transduced with the LVs expressing miR #7, #7mod and #10, confirming the results obtained in K562 cells with these miR #HBBs (FIG. 3A). As expected, the decrease of the endogenous β-globin expression was correlated with the VCN. Indeed, for miR #7, #7mod and #10, we observed a reduction of the β-globin expression ranging from 60% to 85% and from 30% to 70% at a high and low VCN, respectively (FIG. 3B). The miR #1 and #5mod showed almost no effects on β^{AS3} and β-globin expression although we observed a modest β^{AS3}-downregulation in K562 cells. Moreover, we obtained a comparable VCN in cells transduced with control- and therapeutic-LVs at the same MOIs, showing that the introduction of the miR #HBB did not impact gene transfer efficiency. Finally, we analyzed by Western Blot the total β like-globin (β+β^{AS3}) expression and observed 35% silencing of the (β+β^{AS3})-globins at both VCN for miR #7mod and 25% and 10% at high and low VCN, respectively, for miR #10 (FIGS. 3C, 3D). However, we did not observe silencing of the (β+β^{AS3})-globins with miR #7 (FIGS. 3C, 3D). Therefore, in this experiment, the modified version of miR #7 (miR #7mod) outperformed miR #7 in terms of β-like globin silencing at both the RNA and protein levels.

### Modification of the Transgene Sequence and Design of the Novel AS3mod/miR Lentiviral Vectors

Based on our results, we selected miR #7mod and miR #10 as our best performing miRs in terms of β-globin silencing. However, the very high sequence similarity between the β^{S}-globin and the transgene require the modification of the targeted sequence in the transgene to avoid its silencing by the miR. To this aim, we introduced silent mutations in the β^{AS3} transgene sequence in order to maintain the same amino acid sequence. When possible, we chose codons amongst the most used ones in the HBB gene to avoid potential alterations in the translation of the β^{AS3} transcript. In the miR #7mod or miR #10 target regions, we have introduced several mutations to reduce by 33% the complementarity between the miRs and the β^{AS3} transcript.

Then, we designed 3 types of LVs containing the modified β^{AS3} sequences:
The 2 LVs of interest co-expressing the β^{AS3} transgene (modified in the miR #7mod or miR #10 target sequence; AS3mod) and miR #7mod or miR #10 (LV-AS3mod/miR #7mod and LV-AS3mod/miR #10).
Control LVs co-expressing the β^{AS3} transgene (modified in the miR #7mod or miR #10 target sequence) and a control no-targeting miR (LV-AS3mod/miR #nt).
Control LVs expressing only the β^{AS3} transgene (modified in the miR #7mod or miR #10 target sequence) (LV-AS3mod).
   Notably, vector titers were comparable for the control and miR-expressing LVs: neither the transgene modification nor the miR insertion impacted the viral titer.
   Validation of LV-AS3/miR #HBB in Primary Hematopoietic Stem/Progenitor Cells from SCD Patients

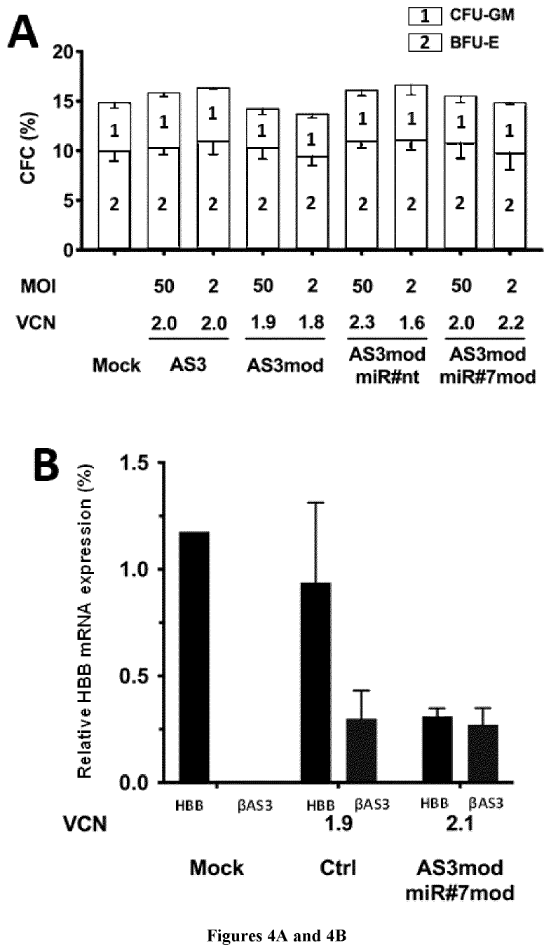

We transduced primary adult hematopoietic stem/progenitor cells (HSPCs) derived from SCD donors with LV-AS3mod/miR #7mod harboring an amiR against HBB. As control LVs, we used LV-AS3mod/miR #nt, LV-AS3mod and the LV-AS3 vector harboring the unmodified AS3 transgene (LV-AS3; (Weber et al., 2018)). Mock- and transduced HSPCs were plated in clonogenic cultures (colony forming cell [CFC] assay) allowing the growth of erythroid (BFU-E) and granulomonocytic (CFU-GM) progenitors. The number and the proportion of BFU-E and CFU-GM was similar amongst the different samples (FIG. 4A), indicating no impairment in erythroid and granulomonocytic cell growth and differentiation. VCN in BFU-E ranged around 2 in all the samples (FIG. 4A).

To evaluate the potential therapeutic effect of this strategy, we measured HBB and β^{AS3}-globin mRNA expression in mock- and LV-transduced erythroid cells (BFU-E) derived from SCD HSPCs. Interestingly, we observed a robust knock-down of HBB expression in samples transduced with LV-AS3mod/miR #7mod, while β^{AS3}-globin mRNA expression was not affected (FIG. 4B). These results were confirmed at protein level by HPLC analysis showing a reduced HbS expression and increased incorporation of the β^{AS3} therapeutic chain into the hemoglobin tetramers (FIG. 4C). Notably, the modification of the AS3 transgene did not affect its expression (LV-AS3mod vs LV-AS3 samples; FIG. 4C).

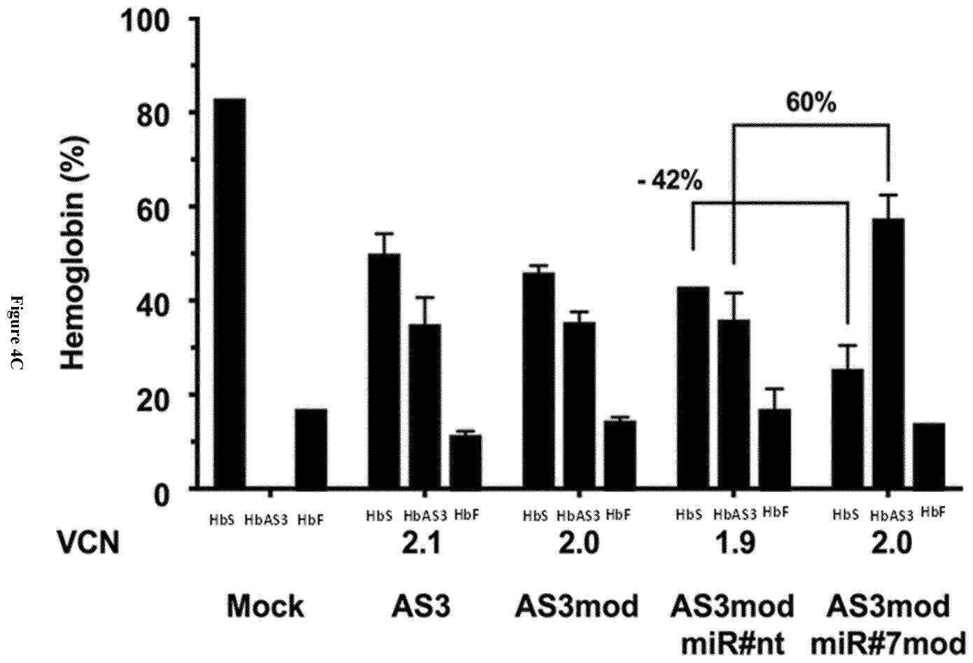

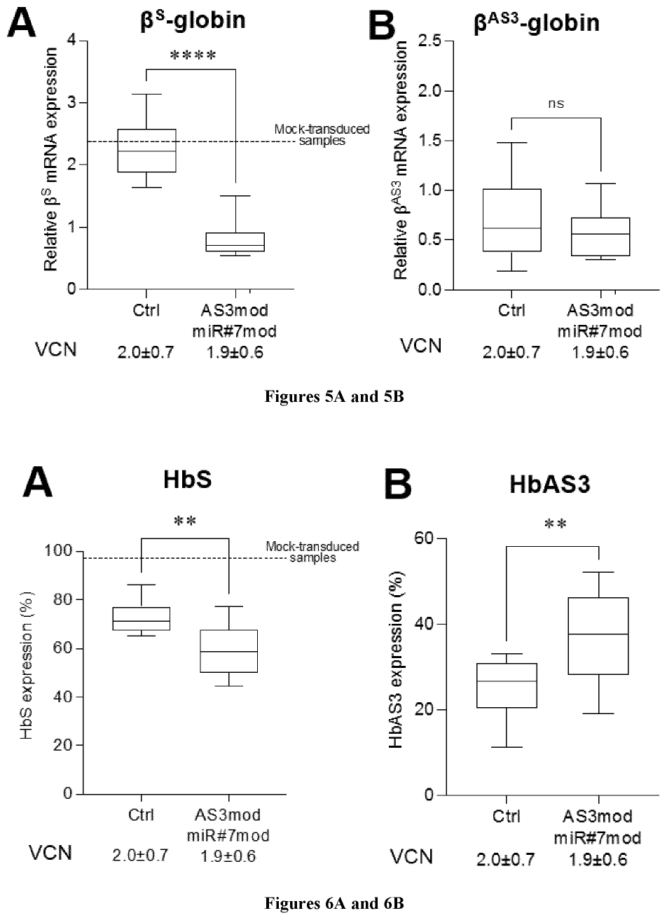

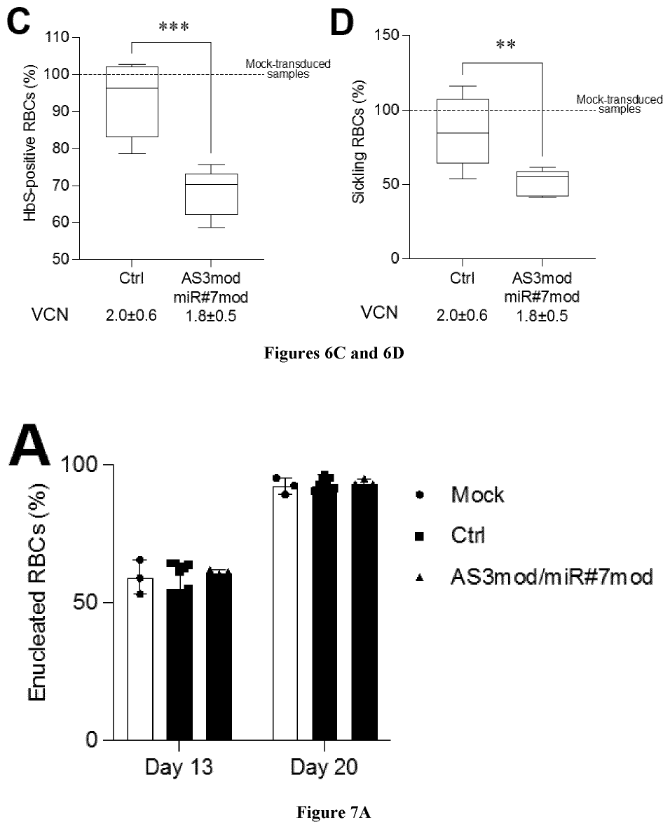

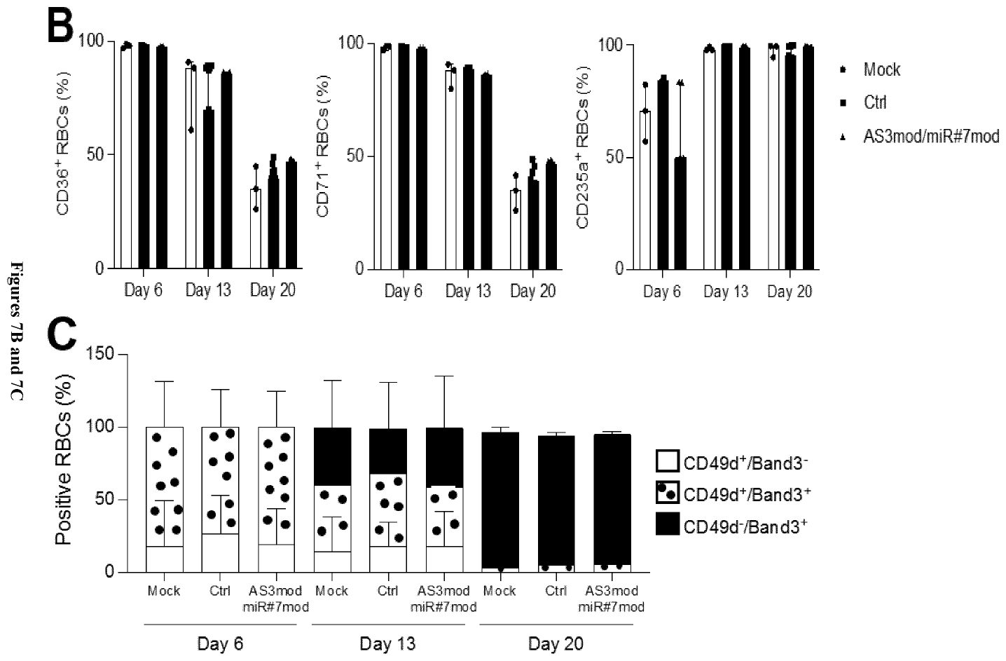

To evaluate the reversion of the SCD cell phenotype, HSPCs from three SCD patients were either mock-transduced or transduced with LV-AS3mod/miR #7mod or LV-control (ctrl, LV-AS3, LV-AS3mod, or LV-AS3mod/miR #nt) vectors and terminally differentiated into mature enucleated RBCs. Efficient HSPC transduction by AS3mod/miR #7mod LV led to a substantial decrease of β^{S}-globin transcripts in HSPC-derived erythroid cells compared to LV-ctrl transduced cells (RTqPCR) at a mean VCN/cell of 2 (FIG. 5A). Notably, the miR specifically down-regulated β^{S}-globin, without affecting β^{AS3} expression (FIG. 5B). In AS3mod/miR #7mod- vs control LV-transduced cells, HPLC analysis showed that β^{S}-globin downregulation led to a significant decrease of HbS, which represented 58% and 71% of the total Hb, respectively; FIG. 6A). This was associated with a significant increase of the therapeutic Hb in AS3mod/miR #7mod LV- compared to ctrl LV-transduced erythroid cells (38% and 27% of the total Hb, respectively; FIG. 6B). Importantly, we observed a substantial reduction of the proportion of HbS-positive cells in AS3mod/miR #7mod- compared to control LV-transduced samples (from 96% to 70%; FIG. 6C). The increased incorporation of β^{AS3} in Hb tetramers and the decrease in β^{S}-globin led to a better correction of the sickling phenotype in mature RBCs derived from HSPCs transduced with AS3mod/miR #7mod LV- compared to control LV (55% and 84% of sickling cells, respectively; FIG. 6D). Importantly, erythroid differentiation was not affected by β^{S}-globin down-regulation (FIGS. 7A-7C). Overall, these results validate the therapeutic potential of these novel bifunctional LVs.

### Summary and Conclusion

In this study, we sought to develop an effective strategy for SCD gene therapy. This new strategy is based on previous work that led to the development of LVs expressing a potent anti-sickling therapeutic globin under the control of the β-globin promoter and key regulatory elements of the β-globin LCR. Some of these vectors have recently been tested in clinical trials and although they have shown encouraging results, they do not always provide a benefit in patients with SCD. One of the major reasons is because the transduction efficiency is not equivalent between the patients' HSCs and in some cases, the VCN of the drug product is <1. In these patients, the outcome is a low level of therapeutic β-globin expression as well as the persistence of an elevated proportion of HbS in RBCs, resulting in a limited efficacy of the treatment. On the other hand, it was observed in SCD patients coinheriting a SCD mutation and a thalassemic trait (β⁰/β^{S}), which decreases β-globin production, that for the same VCN, this type of vector was more effective due to lower HbS levels than in β^{S}/β^{S} patients (Magrin et al., 2019; Ribeil et al., 2017) .

With the objective of proposing an effective LV to treat SCD patients, we are seeking to improve the LV-AS3 vector, which expresses the potent anti-sickling β^{AS3}-globin by adding an RNA interfering function. To do so, a miR #HBB has been inserted in intron 2 of the β^{AS3} transgene in order to decrease the expression of the β^{S}-globin. The reduction of β^{S}-globin levels will increase the incorporation of β^{AS3}-globin in Hb tetramers, which should allow this new vector (LV-AS3/miR #HBB) to correct the SCD phenotype of patients who express high levels of β^{S}-globin. Reducing β^{S}-globin levels will increase the incorporation of β^{AS3}-globin into Hb tetramers, which should allow LV-AS3/miR #HBB to outperform the current vectors in the correction of the SCD phenotype.

Among the 17 LVs-AS3/miR #HBB tested in K562 and HUDEP-2 cells, we have identified LV-AS3/miR#7mod and LV-AS3/miR #10 as the most efficient LVs to decrease β^{S}-globin expression. Before testing these two LVs in patient HSPCs, we modified the sequence of the transgene by introducing silent mutations at the miR #7mod and miR #10 target sequences. Thus, the complementarity between miR #7mod and miR #10 and their target sequence in the transgene is reduced by 33%, which should be enough to avoid targeting and downregulation of the therapeutic β^{AS3} transgene.

Finally, SCD patient HSPCs were transduced with LV-AS3mod/mir #7mod and control LVs. Erythroid cells derived from patient HSPCs transduced with LV-AS3mod/miR #7mod showed a high level of HbAS3 production associated with a reduction of β^{S}-globin chains, which allowed our bifunctional LVs to outperform the current therapeutic vectors in terms of correction of the sickling cell phenotype.

### REFERENCES

Throughout this application, various references describe the state of the art to which this invention pertains. The disclosures of these references are hereby incorporated by reference into the present disclosure.
Adams, F. F., Heckl, D., Hoffmann, T., Talbot, S. R., Kloos, A., Thol, F., Heuser, M., Zuber, J., Schambach, A., Schwarzer, A., 2017. An optimized lentiviral vector system for conditional RNAi and efficient cloning of microRNA embedded short hairpin RNA libraries. Biomaterials 139, 102-115. https://doi.org/10.1016/j.biomaterials.2017.05.032
Akinsheye, I., Alsultan, A., Solovieff, N., Ngo, D., Baldwin, C. T., Sebastiani, P., Chui, D. H. K., Steinberg, M. H., 2011. Fetal hemoglobin in sickle cell anemia. Blood 118, 19-27. https://doi.org/10.1182/blood-2011-03-325258

Amendola, M., Passerini, L., Pucci, F., Gentner, B., Bacchetta, R., Naldini, L., 2009. Regulated and Multiple miRNA and siRNA Delivery Into Primary Cells by a Lentiviral Platform. Molecular Therapy 17, 1039-1052. https://doi.org/10.1038/mt.2009.48
Antoniani, C., Meneghini, V., Lattanzi, A., Felix, T., Romano, O., Magrin, E., Weber, L., Pavani, G., El Hoss, S., Kurita, R., Nakamura, Y., Cradick, T. J., Lundberg, A. S., Porteus, M.,

Amendola, M., El Nemer, W., Cavazzana, M., Mavilio, F., Miccio, A., 2018. Induction of fetal hemoglobin synthesis by CRISPR/Cas9-mediated editing of the human β-globin locus. Blood 131, 1960-1973. https://doi.org/10.1182/blood-2017-10-811505
Brendel, C., Guda, S., Renella, R., Bauer, D. E., Canver, M. C., Kim, Y.-J., Heeney, M. M., Klatt, D., Fogel, J., Milsom, M. D., Orkin, S. H., Gregory, R. I., Williams, D. A., 2016. Lineage-specific BCL11A knockdown circumvents toxicities and reverses sickle phenotype. J Clin Invest 126, 3868-3878. https://doi.org/10.1172/JCI87885
Brendel, C., Negre, O., Rothe, M., Guda, S., Parsons, G., Harris, C., McGuinness, M., Abriss, D., Tsytsykova, A., Klatt, D., Bentler, M., Pellin, D., Christiansen, L., Schambach, A., Manis, J., Trebeden-Negre, H., Bonner, M., Esrick, E., Veres, G., Armant, M., Williams, D. A., 2020. Preclinical Evaluation of a Novel Lentiviral Vector Driving Lineage-Specific BCL11A Knockdown for Sickle Cell Gene Therapy. Molecular Therapy-Methods & Clinical Development 17, 589-600. https://doi.org/10.1016/j.omtm.2020.03.015
Calin, G. A., Dumitru, C. D., Shimizu, M., Bichi, R., Zupo, S., Noch, E., Aldler, H., Rattan, S., Keating, M., Rai, K., Rassenti, L., Kipps, T., Negrini, M., Bullrich, F., Croce, C. M., 2002. Frequent deletions and down-regulation of micro-RNA genes miR15 and miR16 at 13q14 in chronic lymphocytic leukemia. Proc. Natl. Acad. Sci. U.S.A. 99, 15524-15529. https://doi.org/10.1073/pnas.242606799
Canver, M. C., Smith, E. C., Sher, F., Pinello, L., Sanjana, N. E., Shalem, O., Chen, D. D., Schupp, P. G., Vinjamur, D. S., Garcia, S. P., Luc, S., Kurita, R., Nakamura, Y., Fujiwara, Y., Maeda, T., Yuan, G.-C., Feng, Z., Orkin, S. H., Bauer, D. E., 2015. BCL11A enhancer dissection by Cas9-mediated in situ saturating mutagenesis. Nature 527, 192-197. https://doi.org/10.1038/nature15521
Cavazzana, M., Antoniani, C., Miccio, A., 2017. Gene Therapy for β-Hemoglobinopathies. Molecular Therapy 25, 1142-1154. https://doi.org/10.1016/j.ymthe.2017.03.024
Chakraborty, C., Sharma, A. R., Sharma, G., Doss, C. G. P., Lee, S.-S., 2017. Therapeutic miRNA and siRNA: Moving from Bench to Clinic as Next Generation Medicine. Molecular Therapy-Nucleic Acids 8, 132-143. https://doi.org/10.1016/j.omtn.2017.06.005
Esrick, E.B., Achebe, M., Armant, M., Bartolucci, P., Ciuculescu, M. F., Daley, H., Dansereau, C., Di Caprio, G., Goncalves, B., Hebert, N., Heeney, M. M., Higgins, J. M., Lehmann, L. E., Manis, J. P., Negre, 0., Nikiforow, S., Schonbrun, E., Wood, D. K., Williams, D. A., 2019. Validation of BCL11A As Therapeutic Target in Sickle Cell Disease: Results from the Adult Cohort of a Pilot/Feasibility Gene Therapy Trial Inducing Sustained Expression of Fetal Hemoglobin Using Post-Transcriptional Gene Silencing. Blood 134, LBA-5-LBA-5. https://doi. org/10.1182/blood-2019-132745
Franco, R. S., Yasin, Z., Palascak, M. B., Ciraolo, P., Joiner, C. H., Rucknagel, D. L., 2006. The effect of fetal hemoglobin on the survival characteristics of sickle cells. Blood 108, 1073-1076. https://doi.org/10.1182/blood-2005-09-008318
Guda, S., Brendel, C., Renella, R., Du, P., Bauer, D. E., Canver, M. C., Grenier, J. K., Grimson, A. W., Kamran, S. C., Thornton, J., de Boer, H., Root, D. E., Milsom, M. D., Orkin, S. H., Gregory, R.I., Williams, D.A., 2015. miRNA-embedded shRNAs for Lineage-specific BCL11A Knockdown and Hemoglobin F Induction. Molecular Therapy 23, 1465-1474. https://doi.org/10.1038/mt.2015.113
Kurita, R., Suda, N., Sudo, K., Miharada, K., Hiroyama, T., Miyoshi, H., Tani, K., Nakamura, Y., 2013. Establishment of Immortalized Human Erythroid Progenitor Cell Lines Able to Produce Enucleated Red Blood Cells. PLOS ONE 8, e59890. https://doi.org/10.1371/journal.pone.0059890
Lin, S.-L., Ying, S.-Y., 2018. Gene Silencing In Vitro and In Vivo Using Intronic MicroRNAs, in: Ying, S.-Y. (Ed.), MicroRNA Protocols, Methods in Molecular Biology. Springer New York, New York, NY, pp. 107-126. https://doi.org/10.1007/978-1-4939-7601-0_9
Luc, S., Huang, J., McEldoon, J. L., Somuncular, E., Li, D., Rhodes, C., Mamoor, S., Hou, S., Xu, J., Orkin, S. H., 2016. Bc111a-deficiency leads to hematopoietic stem cell defects with an aging-like phenotype. Cell Rep 16, 3181-3194. https://doi.org/10.1016/j.celrep.2016.08.064
Magrin, E., Miccio, A., Cavazzana, M., 2019. Lentiviral and genome-editing strategies for the treatment of β-hemoglobinopathies. Blood 134, 1203-1213. https://doi.org/10.1182/blood.2019000949
Marktel, S., Scaramuzza, S., Cicalese, M. P., Giglio, F., Galimberti, S., Lidonnici, M. R., Calbi, V., Assanelli, A., Bernardo, M. E., Rossi, C., Calabria, A., Milani, R., Gattillo, S., Benedicenti, F., Spinozzi, G., Aprile, A., Bergami, A., Casiraghi, M., Consiglieri, G., Masera, N., D'Angelo, E., Mirra, N., Origa, R., Tartaglione, I., Perrotta, S., Winter, R., Coppola, M., Viarengo, G., Santoleri, L., Graziadei, G., Gabaldo, M., Valsecchi, M. G., Montini, E., Naldini, L., Cappellini, M. D., Ciceri, F., Aiuti, A., Ferrari, G., 2019. Intrabone hematopoietic stem cell gene therapy for adult and pediatric patients affected by transfusion-dependent β-thalassemia. Nat Med 25, 234-241. https://doi.org/10.1038/s41591-018-0301-6
O'Brien, J., Hayder, H., Zayed, Y., Peng, C., 2018. Overview of MicroRNA Biogenesis, Mechanisms of Actions, and Circulation. Front. Endocrinol. 9, 402. https://doi.org/10.3389/fendo.2018.00402
Poletti, V., Charrier, S., Corre, G., Gjata, B., Vignaud, A., Zhang, F., Rothe, M., Schambach, A., Gaspar, H. B., Thrasher, A. J., Mavilio, F., 2018. Preclinical Development of a Lentiviral Vector for Gene Therapy of X-Linked Severe Combined Immunodeficiency. Molecular Therapy—Methods & Clinical Development 9, 257-269. https://doi.org/10.1016/j.omtm.2018.03.002
Powars, D. R., Weiss, J. N., Chan, L. S., Schroeder, W. A., 1984. Is there a threshold level of fetal hemoglobin that ameliorates morbidity in sickle cell anemia? Blood 63,921-926.
Ribeil, J. -A., Hacein-Bey-Abina, S., Payen, E., Magnani, A., Semeraro, M., Magrin, E., Caccavelli, L., Neven, B., Bourget, P., El Nemer, W., Bartolucci, P., Weber, L., Puy, H., Meritet, J. -F., Grevent, D., Beuzard, Y., Chrétien, S., Lefebvre, T., Ross, R. W., Negre, O., Veres, G., Sandler, L., Soni, S., de Montalembert, M., Blanche, S., Leboulch, P., Cavazzana, M., 2017. Gene Therapy in a Patient with Sickle Cell Disease [WWW Document]. http://dx.doi.org/10.1056/NEJMoa1609677. https://doi.org/10.1056/NEJMoa1609677
Sankaran, V. G., Menne, T. F., Xu, J., Akie, T. E., Lettre, G., Handel, B. V., Mikkola, H. K. A., Hirschhorn, J. N., Cantor, A. B., Orkin, S. H., 2008. Human Fetal Hemoglobin Expression Is Regulated by the Developmental Stage-Specific Repressor BCL11A. Science 322,1839-1842. https://doi.org/10.1126/science.1165409
Sundd, P., Gladwin, M. T., Novelli, E. M., 2018. Pathophysiology of Sickle Cell Disease 32. Weber, L., Poletti, V., Magrin, E., Antoniani, C., Martin, S., Bayard, C., Sadek, H., Felix, T., Meneghini, V., Antoniou, M. N., El-Nemer, W., Mavilio, F., Cavazzana, M., Andre-Schmutz, I., Miccio, A., 2018. An Optimized Lentiviral Vector Efficiently Corrects the Human Sickle Cell Disease Phenotype. Molecular Therapy-Methods & Clinical Development 10,268-280. https://doi.org/10.1016/j.omtm.2018.07.012
Yu, Y., Wang, J., Khaled, W., Burke, S., Li, P., Chen, X., Yang, W., Jenkins, N. A., Copeland, N. G., Zhang, S., Liu, P., 2012. Bc111a is essential for lymphoid development and negatively regulates p53. J Exp Med 209,2467-2483. https://doi.org/10.1084/jem.20121846

1. A nucleic acid molecule having the sequence as set forth in SEQ ID NO:1 wherein a sequence encoding for an artificial microRNA (amiR) suitable for reducing the expression of the β^{S}-globin is inserted between the nucleotide at position 85 and the nucleotide at position 86 in SEQ ID NO:1 and/or ii) between the nucleotide at position 146 and the nucleotide at position 147 in SEQ ID NO:1.

2. The nucleic acid molecule of claim 1 wherein the amiR comprises or consists of a shRNA that is embedded into a miRNA backbone and wherein the shRNA adopts a stem-loop structure wherein the stem region is a region formed by a guide strand and a passenger strand.

3. The nucleic acid molecule of claim 2 wherein the miRNA backbone is derived from miR-223.

4. The nucleic acid molecule of claim 2 wherein the sequence encoding for the guide strand comprises or consists of a nucleic acid sequence selected from SEQ ID NO:3 to SEQ ID NO:22.

5. The nucleic acid molecule of claim 4 wherein the sequence encoding for the guide strand comprises or consists of a nucleic acid sequence that is complementary to the nucleic acid sequence as set forth in SEQ ID NO:23 or SEQ ID NO:24.

6. The nucleic acid molecule of claim 4 wherein the sequence encoding for the guide strand comprises or consists of the nucleic acid sequence of SEQ ID NO:15 or SEQ ID NO:18.

7. The nucleic acid molecule of claim 2 wherein the loop segment is encoded by the sequence as set forth in SEQ ID NO:25.

8. The nucleic acid molecule of claim 2 wherein the sequence encoding for the shRNA is selected from SEQ ID NO:26 to SEQ ID NO:45.

9. The nucleic acid molecule of claim 2 wherein the sequence encoding for the shRNA is SEQ ID NO:38 or SEQ ID NO:41.

10. The nucleic acid molecule of claim 2 wherein the sequence encoding for the amiR is a sequence selected from SEQ ID NO:46 to SEQ ID NO:65.

11. The nucleic acid molecule of claim 2 wherein the sequence encoding for the amiR is SEQ ID NO:58 or SEQ ID NO:61.

12. The nucleic acid molecule of claim 2 that has a sequence selected from SEQ ID NO:66 to SEQ ID NO:85.

13. The nucleic acid molecule of claim 2 that has the sequence of SEQ ID NO:78 or SEQ ID NO:81.

14. A transgene encoding for an anti-sickling human hemoglobin subunit beta (HBB), wherein said transgene comprises the nucleic acid molecule of claim 1.

15. The transgene of claim 14 that comprises a least one silent mutation so that the expression of a βAS3 polypeptide is not reduced or silenced by amiR when the transgene is expressed.

16. The transgene of claim 14 which comprises the sequence as set forth in SEQ ID NO:86 or SEQ ID NO:87.

17. A lentiviral vector comprising the transgene of claim 14.

18. A method of obtaining a population of host cells transduced with the transgene of claim 14, which comprises the step of transducing a population of host cells in vitro, ex vivo or in vivo with a lentiviral vector comprising the transgene.

19. The method of claim 18 wherein the host cells are selected from the group consisting of hematopoietic stem/progenitor cells, hematopoietic progenitor cells, hematopoietic stem cells (HSCs), pluripotent cells and induced pluripotent stem cells (iPS).

20. A method of treating sickle cell disease in a subject in need thereof, the method comprising transplanting a therapeutically effective amount of a population of host cells obtained by the method of claim 18.
